(19) United States
(12) Patent Application Publication
(10) Pub. No.: US 2022/0305100 A1
(43) Pub. Date: Sep. 29, 2022

(54) METHODS OF VACCINATION AND USE OF CD47 BLOCKADE

(71) Applicant: DCPRIME B.V., Leiden (NL)

(72) Inventors: Erik Hans Manting, Leiden (NL); Satwinder Kaur Singh, Leiden (NL); Haoxiao Zuo, Leiden (NL)

(21) Appl. No.: 17/692,321

(22) Filed: Mar. 11, 2022

Related U.S. Application Data
(60) Provisional application No. 63/160,296, filed on Mar. 12, 2021.

(51) Int. Cl. A61K 39/00; A61K 39/395; C12N 5/09

(52) U.S. Cl. CPC A61K 39/0011; A61K 39/3955; C12N 5/0694; C12N 2510/00; A61K 2039/5152; A61K 2039/545; A61K 2039/552

(57) ABSTRACT

The present disclosure provides a modified cell of leukemic origin comprising a downregulated CD47 pathway. Methods for using the modified cells in treating cancer alone, or in combination with CD47 blockade are also provided. Also provided are compositions comprising a modified cell of leukemic origin, pharmaceutical compositions and formulations thereof, and methods of producing the modified cells.

### RELATED APPLICATIONS

This application claims the benefit of U.S. Provisional Application No. 63/160,296, filed Mar. 12, 2021, which is incorporated by reference in its entirety for all purposes.

### BACKGROUND

Activating the immune system to target and kill cancer cells to produce clinically relevant responses has been the aim of cancer research. Immunotherapy aims to incite robust immune response against cancers by targeting molecules expressed on immune cells and cancers. Immense effort has been invested in developing effective cancer vaccines by identifying tumor-specific antigens. Cancer vaccines are designed to boost the immune system's ability to target and destroy such antigens and the cells that display them.

However, due to the complexity of tumor escape mechanisms, conventional cancer vaccines do not always provide a desired immune response in every patient, thereby decreasing the efficacy of immunotherapy. Further, immune checkpoints present a variety of inhibitory barriers that control the immune system, for which tumors have been shown to regulate as a mechanism to develop immune resistance. For example, the first immune checkpoint was discovered when T cells were observed to be controlled by a negative immune checkpoint protein called CTLA-4. CTLA-4 was found to shut down the activity of T cells to prevent them from accidentally damaging healthy cells.

Hence, there is a need in the art for novel immunotherapeutic approaches to treat cancer. In particular, there is a need for novel cancer vaccination approaches to treat cancer. The present invention addresses and satisfies this need.

### SUMMARY

The present disclosure is based, at least in part, on the finding that certain leukemia-derived cells (e.g., a modified cell of leukemic origin as described herein) are efficiently processed by antigen presenting cells. This supports a mechanism whereby when such leukemia-derived cells are administered to a subject as a cell-based vaccine, the dendritic cells of the subject are involved in the digestion and processing of antigens carried by the leukemia-derived cells and subsequent stimulation of the local and systemic immune response. Based on the immune stimulatory capacity of the leukemia-derived cell-based vaccine, after administration of the vaccine into the skin, the intratumoral microenvironment, or other vaccination sites, the vaccine may induce an immunogenic environment by recruiting immune cells. Recruitment of the immune cells to the site of vaccine administration induces the secretion of cytokines and stimulation of phagocytosis of vaccine components by resident and recruited antigen presenting cells.

As described herein, the leukemia-derived cell-based vaccine phagocytosis process has been found to be regulated by SIRPα/CD47 pathways which provide a prominent “do not eat me” signal limiting the interaction between the leukemia-derived cell-based vaccine and antigen-presenting cells. Use of an anti-CD47 blocking antibody was found to enhance the uptake of leukemia-derived cells by antigen presenting cells. This may demonstrate a mode of action in which the cell-based vaccine and CD47 blocking agent function in a synergistic manner by exposing the tumor to the immune system and further boosting the biological activity of the leukemia-derived cell vaccine.

An exemplary leukemia-derived cell vaccine of the invention is DCP-001. DCP-001 is a vaccine derived from the DCOne leukemic cell line, DCOne cells of which can adopt a highly immunogenic mature dendric cell (mDC) phenotype. DCOne cells express multiple common tumor-associated antigens. DCOne mDC combine the DCOne tumor-associated antigen repertoire with a mDC costimulatory profile and form the basis for DCP-001, a non-proliferating, frozen, irradiated product. As described herein, DCP-001 is unexpectedly efficacious in the treatment of solid tumor cancers (e.g., a non-leukemia cancer) despite being of a different origin than the leukemic origin from which DCP-001 is derived.

The present disclosure describes the use of a cell based vaccine that has been derived from a cancer (e.g., DCP-001). While current developments in CD47 immunotherapy are directed to blocking CD47 on the surface of cancer cells, the present disclosure relates to CD47 immunotherapy that is directed to blocking CD47 on the surface of cells used to treat a cancer. For example, the blockade of CD47 in the present disclosure is, in certain embodiments, directed to blocking CD47 on the surface of the cells of a cell-based vaccine (e.g., DCP-001).

In one aspect, a pharmaceutical composition comprising an isolated modified cell of leukemic origin comprising a downregulated CD47 pathway, and a pharmaceutically acceptable excipient, is provided.

In certain exemplary embodiments, the downregulated CD47 pathway is a result of the depletion and/or inhibition of a member of the CD47 pathway. In certain exemplary embodiments, the member of the CD47 pathway is CD47. In certain exemplary embodiments, the downregulated CD47 pathway is the result of the depletion and/or inhibition of CD47 and/or a member of the CD47 pathway.

In certain exemplary embodiments, the downregulated CD47 pathway is mediated by an agent that depletes and/or inhibits CD47 and/or a member of the CD47 pathway. In certain exemplary embodiments, the agent that depletes CD47 and/or a member of the CD47 pathway is selected from the group consisting of an antibody, a small molecule, a small RNA, or an engineered nuclease system. In certain exemplary embodiments, the antibody is an anti-CD47 antibody. In certain exemplary embodiments, the small RNA is a small interfering RNA (siRNA) or a microRNA (miRNA). In certain exemplary embodiments, the engineered nuclease system is selected from the group consisting of a meganuclease system, a zinc finger nuclease (ZFN) system, a Transcription activator-like effector nuclease (TALEN) system, and a CRISPR system. In certain exemplary embodiments, the engineered nuclease system mediates an insertion and/or deletion in a CD47 gene locus and/or the gene locus of a member of the CD47 pathway of the modified cell. In certain exemplary embodiments, the engineered nuclease system mediates an insertion and/or deletion in a CD47 gene locus of the modified cell. In certain exemplary embodiments, the engineered nuclease system is a CRISPR system.

In another aspect, a pharmaceutical composition comprising a modified cell of leukemic origin comprising an insertion and/or deletion in a CD47 gene locus, wherein the insertion and/or deletion in the CD47 gene locus results in downregulated expression of CD47, is provided.

In certain exemplary embodiments, the insertion and/or deletion in a CD47 gene locus is mediated by the repair of a double strand break in the CD47 gene locus. In certain exemplary embodiments, the repair is via non-homologous end joining (NHEJ) and homology directed repair (HDR).

In certain exemplary embodiments, the insertion and/or deletion in the CD47 gene locus is mediated by an engineered nuclease system. In certain exemplary embodiments, the engineered nuclease system is selected from the group consisting of a meganuclease system, a zinc finger nuclease (ZFN) system, a Transcription activator-like effector nuclease (TALEN) system, and a CRISPR system. In certain exemplary embodiments, the engineered nuclease system is a CRISPR system.

In certain exemplary embodiments, the pharmaceutical composition further comprises an agent that depletes and/or inhibits CD47 and/or a member of the CD47 pathway. In certain exemplary embodiments, the agent that depletes and/or inhibits CD47 and/or a member of the CD47 pathway is an anti-CD47 antibody.

In another aspect, a pharmaceutical composition comprising a modified cell of leukemic origin and an anti-CD47 antibody, is provided.

In another aspect, a pharmaceutical composition comprising a modified cell of leukemic origin comprising a downregulated CD47 pathway and an anti-CD47 antibody, is provided.

In certain exemplary embodiments, the pharmaceutical composition further comprises a pharmaceutically acceptable excipient. In certain exemplary embodiments, the pharmaceutical composition further comprises a cryopreservation agent.

In certain exemplary embodiments, the modified cell comprises at least one tumor associated antigen or a nucleic acid encoding at least one tumor associated antigen, wherein the tumor associated antigen is selected from the group consisting of WT-1, MUC-1, RHAMM, PRAME, p53, and Survivin. In certain exemplary embodiments, the modified cell comprises WT-1, MUC-1, PRAME, and Survivin. In certain exemplary embodiments, the modified cell comprises an exogenous antigen. In certain exemplary embodiments, the exogenous antigen is a tumor-associated antigen. In certain exemplary embodiments, the modified cell comprises a dendritic cell phenotype. In certain exemplary embodiments, the modified cell comprises a mature dendritic cell phenotype. In certain exemplary embodiments, the modified cell comprises a genetic aberration between chromosome 11p15.5 to 11p12. In certain exemplary embodiments, the genetic aberration encompasses about 16 Mb of genomic regions. In certain exemplary embodiments, the modified cell is CD34-positive, CD1a-positive, and

CD83-positive. In certain exemplary embodiments, the modified cell expresses a cell surface marker selected from the group consisting of DC-SIGN, Langerin, CD80, CD86, CD70, CD40, and any combination thereof. In certain exemplary embodiments, the modified cell is CD34-positive, CD1a-positive, CD83-positive, CD80-positive, CD86-positive, and CD40-positive. In certain exemplary embodiments, the modified cell is CD14-negative. In certain exemplary embodiments, the modified cell is derived from the DCOne cell line. In certain exemplary embodiments, the modified cell is non-proliferating. In certain exemplary embodiments, the modified cell has been irradiated.

In another aspect, a method of producing a modified cell of leukemic origin comprising a downregulated CD47 pathway, comprising: incubating a precursor cell under conditions that allow for the differentiation of the precursor cell into an immature cell; and incubating the immature cell in the presence of an agent that depletes and/or inhibits CD47 and/or a member of the CD47 pathway, and under conditions that allows for the maturation of the immature cell, thereby producing the modified cell comprising a downregulated CD47 pathway, is provided.

In certain exemplary embodiments, the agent that depletes and/or inhibits CD47 and/or a member of the CD47 pathway is selected from the group consisting of an antibody, a small molecule, a small RNA, or an engineered nuclease system. In certain exemplary embodiments, the antibody is an anti-CD47 antibody. In certain exemplary embodiments, the small RNA is a small interfering RNA (siRNA) or a microRNA (miRNA).

In certain exemplary embodiments, the engineered nuclease system mediates an insertion and/or deletion in a CD47 gene locus and/or the gene locus of a member of the CD47 pathway of the modified cell. In certain exemplary embodiments, the engineered nuclease system mediates an insertion and/or deletion in a CD47 gene locus of the modified cell. In certain exemplary embodiments, the engineered nuclease system is selected from the group consisting of a meganuclease system, a zinc finger nuclease (ZFN) system, a Transcription activator-like effector nuclease (TALEN) system, and a CRISPR system. In certain exemplary embodiments, the engineered nuclease system is a CRISPR system.

In another aspect, a pharmaceutical composition comprising the modified cell produced by any of the foregoing methods, is provided.

In certain exemplary embodiments, the pharmaceutical composition further comprises a pharmaceutically acceptable excipient. In certain exemplary embodiments, the pharmaceutical composition further comprises a cryopreservation agent.

In another aspect, a method of enhancing an immune response in a subject in need thereof, comprising administering to the subject an effective amount of any of the foregoing compositions, is provided. In another aspect, any of the foregoing compositions for use in a method of enhancing an immune response in a subject in need thereof, is provided.

In another aspect, a method of treating or preventing cancer in a subject in need thereof, comprising administering to the subject an effective amount of any of the foregoing compositions, is provided. In another aspect, any of the foregoing compositions for use in a method of treating or preventing cancer in a subject in need thereof, is provided.

In another aspect, a method of enhancing an immune response in a subject in need thereof, comprising administering to the subject a first composition comprising a modified cell of leukemic origin comprising a downregulated CD47 pathway, is provided.

In certain exemplary embodiments, the first composition further comprises an agent that depletes and/or inhibits CD47 and/or a member of the CD47 pathway.

In certain exemplary embodiments, the method further comprises administering to the subject an effective amount of a second composition comprising an agent that depletes and/or inhibits CD47.

In certain exemplary embodiments, the first composition and the second composition are administered simultaneously. In certain exemplary embodiments, the first composition is administered before the second composition. In certain exemplary embodiments, the first composition is administered after the second composition.

In another aspect, a method of enhancing an immune response in a subject in need thereof, comprising administering to the subject a first composition comprising a modified cell of leukemic origin, and an agent that depletes and/or inhibits CD47 and/or a member of the CD47 pathway, is provided.

In certain exemplary embodiments, the modified cell comprises at least one tumor associated antigen or a nucleic acid encoding the tumor associated antigen, wherein the tumor associated antigen is associated with the tumor in the subject. In certain exemplary embodiments, the modified cell comprises at least one tumor associated antigen or a nucleic acid encoding the tumor associated antigen, wherein the tumor associated antigen is not associated with the tumor in the subject.

In certain exemplary embodiments, the first composition and/or the second composition is administered via a route selected from the group consisting of intramuscular, subcutaneous, intravenous, intraarterial, intraperitoneal, intrasternal, intradermal, transcutaneous, transdermal, delivery to the interstitial space of a tissue, and delivery to a non-tumor tissue.

In certain exemplary embodiments, the first composition and/or the second composition is administered intravenously. In certain exemplary embodiments, the first composition and/or the second composition is prepared for intravenous administration. In certain exemplary embodiments, the first composition and/or the second composition comprises a diluent or solvent acceptable for intravenous administration.

In certain exemplary embodiments, the first composition and/or the second composition is administered intradermally. In certain exemplary embodiments, the first composition and/or the second composition is prepared for intradermal administration. In certain exemplary embodiments, the first composition and/or the second composition comprises a diluent or solvent acceptable for intradermal administration.

In certain exemplary embodiments, the first composition and/or the second composition is administered intramuscularly. In certain exemplary embodiments, the first composition and/or the second composition is prepared for intramuscular administration. In certain exemplary embodiments, the first composition and/or the second composition comprises a diluent or solvent acceptable for intramuscular administration.

In certain exemplary embodiments, the first composition and/or the second composition is administered intratumorally. In certain exemplary embodiments, the first composition and/or the second composition is prepared for intratumoral administration. In certain exemplary embodiments, the first composition and/or the second composition comprises a diluent or solvent acceptable for intratumoral administration.

In certain exemplary embodiments, the agent that depletes and/or inhibits CD47 is selected from the group consisting of an antibody, a small molecule, a small RNA, or an engineered nuclease system. In certain exemplary embodiments, the antibody is an anti-CD47 antibody. In certain exemplary embodiments, the small RNA is a small interfering RNA (siRNA) or a microRNA (miRNA).

In certain exemplary embodiments, the engineered nuclease system mediates an insertion and/or deletion in a CD47 gene locus and/or the gene locus of a member of the CD47 pathway of the modified cell. In certain exemplary embodiments, the engineered nuclease system mediates an insertion and/or deletion in a CD47 gene locus of the modified cell. In certain exemplary embodiments, the engineered nuclease system is selected from the group consisting of a meganuclease system, a zinc finger nuclease (ZFN) system, a Transcription activator-like effector nuclease (TALEN) system, and a CRISPR system. In certain exemplary embodiments, the engineered nuclease system is a CRISPR system.

In certain exemplary embodiments, the agent that depletes and/or inhibits CD47 comprises a viral vector comprising a nucleic acid encoding an anti-CD47 antibody, a CD47-targeting small RNA, or a CD47-targeting engineered nuclease system. In certain exemplary embodiments, the viral vector is derived from a virus selected from the group consisting of a retrovirus, an adenovirus, an adeno-associated virus, and a herpes simplex virus. In certain exemplary embodiments, the CD47-targeting small RNA is a small interfering RNA (siRNA) or a microRNA (miRNA). In certain exemplary embodiments, the CD47-targeting engineered nuclease system mediates an insertion and/or deletion in a CD47 gene locus of the modified cell.

In certain exemplary embodiments, the CD47-targeting engineered nuclease system is selected from the group consisting of a meganuclease system, a zinc finger nuclease (ZFN) system, a Transcription activator-like effector nuclease (TALEN) system, and a CRISPR system. In certain exemplary embodiments, the CD47-targeting engineered nuclease system is a CRISPR system.

In certain exemplary embodiments, the modified cell comprises at least one tumor associated antigen or a nucleic acid encoding at least one tumor associated antigen, wherein the tumor associated antigen is selected from the group consisting of WT-1, MUC-1, RHAMM, PRAME, p53, and Survivin. In certain exemplary embodiments, the modified cell comprises WT-1, MUC-1, PRAME, and Survivin. In certain exemplary embodiments, the modified cell comprises an exogenous antigen. In certain exemplary embodiments, the exogenous antigen is a tumor-associated antigen. In certain exemplary embodiments, the modified cell comprises a dendritic cell phenotype. In certain exemplary embodiments, the modified cell comprises a mature dendritic cell phenotype. In certain exemplary embodiments, the modified cell comprises a genetic aberration between chromosome 11p15.5 to 11p12. In certain exemplary embodiments, the genetic aberration encompasses about 16 Mb of genomic regions. In certain exemplary embodiments, the modified cell is CD34-positive, CD1a-positive, and CD83-positive. In certain exemplary embodiments, the modified cell expresses a cell surface marker selected from the group consisting of DC-SIGN, Langerin, CD80, CD86, CD70, CD40, and any combination thereof. In certain exemplary embodiments, the modified cell is CD34-positive, CD1a-positive, CD83-positive, CD80-positive, CD86-positive, and CD40-positive. In certain exemplary embodiments, the modified cell is CD14-negative. In certain exemplary embodiments, the modified cell is derived from the DCOne cell line. In certain exemplary embodiments, the modified cell is non-proliferating. In certain exemplary embodiments, the modified cell has been irradiated.

In certain exemplary embodiments, the subject has previously suffered from the cancer. In certain exemplary embodiments, the subject has previously received treatment for the cancer. In certain exemplary embodiments, the subject is suffering from relapse of the cancer.

In certain exemplary embodiments, the cancer is a tumor. In certain exemplary embodiments, the tumor is a solid tumor. In certain exemplary embodiments, the solid tumor is selected from the group consisting of a sarcoma, a carcinoma, and a lymphoma.

In certain exemplary embodiments, the subject is a human. In certain exemplary embodiments, the subject is a domesticated animal and/or an animal suitable for veterinary healthcare.

### BRIEF DESCRIPTION OF THE DRAWINGS

The foregoing and other features and advantages of the present invention will be more fully understood from the following detailed description of illustrative embodiments taken in conjunction with the accompanying drawings.

FIG. 1 is a graph depicting the percentage uptake of DCP-001 or DCOne progenitors (prog) by iMoDCs.

FIG. 2 is a graph depicting the percentage uptake of DCP-001 or DCOne progenitors by specific subpopulation of cells found in PBMCs, as indicated.

FIG. 3 is a graph depicting the percentage uptake of DCP-001 or DCOne progenitors (prog) by iMoDCs when co-cultured in the presence of an agent, as indicated.

FIG. 4A-FIG. 4C are graphs depicting the expression of phosphatidylserine (PS; FIG. 4A), calreticulin (CRT; FIG. 4B) or CD47 (FIG. 4B) on the surface of DCP-001 or DCOne progenitors (prog) as determined by flow cytometry.

FIG. 5A-FIG. 5C are graphs depicting the percentage uptake of DCP-001 or DCOne progenitors (prog) by iMoDCs when co-cultured in the presence of Annexin V (FIG. 5A), a CRT-specific antibody (FIG. 5B), or an anti-CD47 monoclonal antibody (FIG. 5C).

FIG. 6A-FIG. 6H are graphs depicting the secretion of various proinflammatory cytokines and chemokines in PBMC, as indicated, upon stimulation by DCP-001.

### DETAILED DESCRIPTION

Anti-CD47 immunotherapy has been the target of recent research with the aim of blocking CD47 on the surface of cancer cells in order to trigger the phagocytic function of immune cells to engulf the cancer cells. See, e.g., Lu et al., Onco. Targets Ther. (2020) 13: 9323-9331, the disclosure of which is incorporated by reference herein in its entirety. As described herein, the leukemia-derived cell-based vaccine (e.g., DCP-001) phagocytosis process has been found to be regulated by SIRPα/CD47 pathways which provide a prominent “do not eat me” signal limiting the interaction between the leukemia-derived cell-based vaccine and antigen-presenting cells. Use of an anti-CD47 blocking antibody was found to enhance the uptake of DCP-001 by antigen presenting cells. This may demonstrate a mode of action in which the cell-based vaccine and CD47 blocking agent function in a synergistic manner by exposing the tumor to the immune system and further boosting the biological activity of the DCP-001 vaccine.

It is to be understood that the methods described herein are not limited to particular methods and experimental conditions disclosed herein as such methods and conditions may vary. It is also to be understood that the terminology used herein is for the purpose of describing particular embodiments only, and is not intended to be limiting. The methods described herein use conventional molecular and cellular biological and immunological techniques that are well within the skill of the ordinary artisan. Such techniques are well-known to the skilled artisan and are explained in the scientific literature.

### A. DEFINITIONS

Unless otherwise defined, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

Generally, nomenclature used in connection with cell and tissue culture, molecular biology, immunology, microbiology, genetics and protein and nucleic acid chemistry and hybridization described herein is well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

That the disclosure may be more readily understood, select terms are defined below.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, ±5%, ±1%, and ±0.1% from the specified value, as such variations are appropriate to perform the disclosed methods.

As used herein, to “alleviate” a disease means reducing the severity of one or more symptoms of the disease.

The term “antigen” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen.

The term “antigen” or “antigenic,” as used in relation to a polypeptide as described herein, refers generally to a biological molecule which contains at least one epitope specifically recognized by a T-cell receptor, an antibody, or other elements of specific humoral and/or cellular immunity. The whole molecule may be recognized, or one or more portions of the molecule, for instance following intracellular processing of a polypeptide into an MHC peptide antigen complex and subsequent antigen presentation. The term “antigen” or “antigenic” includes reference to at least one, or more, antigenic epitopes of a polypeptide as described herein.

Furthermore, antigens can be derived from recombinant or genomic DNA. A skilled artisan will understand that any DNA, which comprises a nucleotide sequence or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. Moreover, a skilled artisan will understand that an antigen need not be encoded by a “gene” at all. It is readily apparent that an antigen can be generated, synthesized, or can be derived from a biological sample. Such a biological sample can include, but is not limited to a tissue sample, a tumor sample, a cell or a biological fluid.

As used herein, the term “allogeneic” refers to the involvement of living tissues or cells that are genetically dissimilar and hence immunologically incompatible, with respect to a subject in need of treatment. While genetically dissimilar, an allogeneic cell, e.g., an allogeneic leukemia-derived cell (e.g., a modified cell of leukemic origin) described herein, is derived from the same species. For example, a method described herein comprising administering to a subject a modified cell of leukemic origin, refers to the administration of a modified cell of leukemic origin that is genetically dissimilar to the subject, albeit still of the same species.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to an amount that when administered to a mammal, causes a detectable level of immune suppression or tolerance compared to the immune response detected in the absence of the composition of the invention. The immune response can be readily assessed by a plethora of art-recognized methods. The skilled artisan would understand that the amount of the composition administered herein varies and can be readily determined based on a number of factors such as the disease or condition being treated, the age and health and physical condition of the mammal being treated, the severity of the disease, the particular compound being administered, and the like.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

As used herein, the term “exogenous” refers to any material introduced from or produced outside an organism, cell, tissue or system.

The term “subject,” as used herein, refers to the recipient of a method as described herein, i.e., a recipient that can mount a cellular immune response, and is a mammal. In certain embodiments, the subject is a human. In certain embodiments, the subject is a domesticated animal, e.g., a horse, a cow, a pig, a sheep, a dog, a cat, etc. The terms “patient” and “subject” may be used interchangeably. In certain embodiments, the subject is a human suffering from a cancer (e.g., a solid tumor). In certain embodiments, the subject is a domesticated animal suffering from a cancer (e.g., a solid tumor).

The term “therapeutic” as used herein means a treatment and/or prophylaxis. A therapeutic effect is obtained by suppression, remission, or eradication of a disease state.

To “treat” a disease as the term is used herein, means to reduce the frequency or severity of at least one sign or symptom of a disease or disorder experienced by a subject.

The term “tumor,” as used herein, includes reference to cellular material, e.g., a tissue, proliferating at an abnormally high rate. A growth comprising neoplastic cells is a neoplasm, also known as a “tumor,” and generally forms a distinct tissue mass in a body of a subject. A tumor may show partial or total lack of structural organization and functional coordination with the normal tissue. As used herein, a tumor is intended to encompass hematopoietic tumors as well as solid tumors. In certain embodiments, the tumor is a solid tumor. The term “tumor,” as used herein, includes reference to the tumor micro-environment or tumor site, i.e., the area within the tumor and the area directly outside the tumorous tissue. In certain embodiments, the tumor micro-environment or tumor site includes an area within the boundaries of the tumor tissue. In certain embodiments, the tumor micro-environment or tumor site includes the tumor interstitial compartment of a tumor, which is defined herein as all that is interposed between the plasma membrane of neoplastic cells and the vascular wall of the newly formed neovessels. As used herein, the terms “tumor micro-environment” or “tumor site” refers to a location within a subject in which a tumor resides, including the area immediately surrounding the tumor.

A tumor may be benign (e.g., a benign tumor) or malignant (e.g., a malignant tumor or cancer). Malignant tumors can be broadly classified into three major types: those arising from epithelial structures are called carcinomas, those that originate from connective tissues such as muscle, cartilage, fat or bone are called sarcomas, and those affecting hematopoietic structures (structures pertaining to the formation of blood cells) including components of the immune system, are called leukemias and lymphomas. Other tumors include, but are not limited to, neurofibromatosis.

Solid tumors are abnormal masses of tissue that can be benign or malignant. In certain embodiments, solid tumors are named for the type of cells that form them (such as sarcomas, carcinomas, and lymphomas). Examples of solid tumors, such as sarcomas and carcinomas, include, but are not limited to, liposarcoma, fibrosarcoma, chondrosarcoma, osteosarcoma, myxosarcoma, and other sarcomas, mesothelioma, synovioma, leiomyosarcoma, Ewing's tumor, colon carcinoma, rhabdomyosarcoma, pancreatic cancer, lymphoid malignancy, lung cancers, breast cancer, prostate cancer, ovarian cancer, hepatocellular carcinoma, adenocarcinoma, basal cell carcinoma, sweat gland carcinoma, squamous cell carcinoma, medullary thyroid carcinoma, pheochromocytomas sebaceous gland carcinoma, papillary thyroid carcinoma, papillary adenocarcinomas, papillary carcinoma, medullary carcinoma, bronchogenic carcinoma, hepatoma, renal cell carcinoma, bile duct carcinoma, Wilms' tumor, choriocarcinoma, cervical cancer, seminoma, testicular tumor, bladder carcinoma, melanoma, CNS tumors (e.g., a glioma, e.g., brainstem glioma and mixed gliomas, glioblastoma (e.g., glioblastoma multiforme), germinoma, astrocytoma, craniopharyngioma, medulloblastoma, ependymoma, Schwannoma, CNS lymphoma, acoustic neuroma, pinealoma, hemangioblastoma, meningioma, oligodendroglioma, retinoblastoma, neuroblastoma, and brain metastases), and the like.

Carcinomas that can be amenable to therapy by a method disclosed herein include, but are not limited to, squamous cell carcinoma (various tissues), basal cell carcinoma (a form of skin cancer), esophageal carcinoma, bladder carcinoma, including transitional cell carcinoma (a malignant neoplasm of the bladder), hepatocellular carcinoma, colorectal carcinoma, bronchogenic carcinoma, lung carcinoma, including small cell carcinoma and non-small cell carcinoma of the lung, colon carcinoma, thyroid carcinoma, gastric carcinoma, breast carcinoma, ovarian carcinoma, adrenocortical carcinoma, pancreatic carcinoma, sweat gland carcinoma, prostate carcinoma, papillary carcinoma, adenocarcinoma, sebaceous gland carcinoma, medullary carcinoma, papillary adenocarcinoma, ductal carcinoma in situ or bile duct carcinoma, cystadenocarcinoma, renal cell carcinoma, choriocarcinoma, Wilm's tumor, seminoma, embryonal carcinoma, cervical carcinoma, testicular carcinoma, nasopharyngeal carcinoma, osteogenic carcinoma, epithelial carcinoma, uterine carcinoma, and the like.

Sarcomas that can be amenable to therapy by a method disclosed herein include, but are not limited to, myxosarcoma, chondrosarcoma, chordoma, osteogenic sarcoma, liposarcoma, fibrosarcoma, angiosarcoma, lymphangiosarcoma, endotheliosarcoma, osteosarcoma, mesothelioma, Ewing's sarcoma, leiomyosarcoma, rhabdomyosarcoma, lymphangioendotheliosarcoma, synovioma, and other soft tissue sarcomas.

The term “immunogenic composition,” as used herein, refers to a substance which induces a specific immune response against an immunogen in a subject who is in need of an immune response against said immunogen. The composition may include an adjuvant and optionally one or more pharmaceutically-acceptable carriers, excipients and/or diluents. In certain embodiments, the immunogenic composition comprises a modified cell of leukemic origin as described herein.

The term “immune response,” as used herein, includes T-cell mediated and/or B-cell mediated immune responses. Exemplary immune functions of T cells include, e.g., cytokine production and induction of cytotoxicity in other cells. B-cell functions include antibody production. In addition, the term includes immune responses that are indirectly affected by T-cell activation, e.g., antibody production and activation of cytokine responsive cells, e.g., macrophages. Immune cells involved in the immune response include lymphocytes, such as B cells and T cells (CD4⁺ and CD8⁺ cells); antigen presenting cells (e.g., professional antigen presenting cells such as dendritic cells, macrophages, B lymphocytes, Langerhans cells, and non-professional antigen presenting cells such as keratinocytes, endothelial cells, astrocytes, fibroblasts, oligodendrocytes); natural killer cells; myeloid cells, such as macrophages, eosinophils, mast cells, basophils, and granulocytes. In certain embodiments, the term refers to a T-cell mediated immune response. The immune response may in some embodiments be a T cell-dependent immune response.

The term “intratumoral,” as used herein, refers to delivery or transport of material (e.g., a modified cell of leukemic origin) into a tumor. One example of intratumoral delivery, as described herein is by intratumoral administration, a route of administration generally known in the art. As an alternative route for intratumoral administration, the material may be delivered to the tumor via a tumor-specific carrier, such as an oncolytic virus or a gene therapy vector, which have been broadly developed to deliver gene sequences to tumors. The use of such vehicles allows for multiple routes of administration, in addition to intratumoral administration, such by as intravenous or intraperitoneal administration, subsequently resulting in the delivery of the nucleic acid encoding said polypeptide, into the tumor See, e.g., Lundstrom, Diseases, 6(2):42 (2018); Alemany, Biomedicines, 2, p. 36-49 (2014); Twumasi-Boateng et al., Nature Reviews Cancer 18, p. 419-432 (2018), the disclosures of which are incorporated by reference herein in their entireties.

As used herein, the term “extratumoral” refers to a location, e.g., in the body of a subject, that is away (e.g., distal) from a tumor.

The term “modified cell of leukemic origin,” as used herein, refers to a cell derived from a leukemia cell line that can take up an antigen, and present the antigen, or an immunogenic portion thereof together with an MHC class I complex or MHC class II complex. In certain embodiments, the modified cell of leukemic origin is a cell derived from cell line DCOne as deposited under the conditions of the Budapest treaty with the DSMZ under accession number DSMZ ACC3189 on 15 Nov. 2012. The process of obtaining mature cells from the deposited DCOne cell line is for instance described in EP2931878B1, the disclosure of which is incorporated by reference herein in its entirety.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### B. MODIFIED CELL OF LEUKEMIC ORIGIN AND METHODS OF PRODUCTION

Provided herein are methods comprising the use of a modified cell of leukemic origin to stimulate and expand immune cells, generate antigen-specific immune cells, and for methods of treatment. As used herein, the term “modified cell of leukemic origin” refers to a cell capable of taking up an antigen such as an antigenic polypeptide, and capable of presenting the antigen, or an immunogenic part thereof, together with an MHC class I complex or MHC class II complex. A modified cell of leukemic origin provided herein comprises a mature dendritic cell phenotype. The term “dendritic cell,” as used herein, refers to a professional antigen presenting cell (APC) that can take up an antigen such as an antigenic polypeptide into its cell, and presents the antigen, or an immunogenic part thereof together with an MHC class I complex or MHC class II complex. Having a mature dendritic cell phenotype means that the modified cell of leukemic origin is capable of performing similar functions to those of a mature dendritic cell. The term includes both immature dendritic cells (“imDC”) and mature dendritic cells (“mDC”), depending on maturity.

In certain embodiments, the modified cell of leukemic origin is derived from leukemia cells. In certain embodiments, the modified cell of leukemic origin is derived from a patient having leukemia. In certain embodiments, the modified cell of leukemic origin is derived from the peripheral blood of a patient having leukemia. In certain embodiments, the modified cell of leukemic origin is derived from the peripheral blood of a patient having acute myeloid leukemia. The skilled artisan will recognize that a modified cell of leukemic origin can be derived from any patient obtained peripheral blood, wherein the patient has any type of leukemia, given that the modified cell of leukemic origin thus derived comprises the characteristics disclosed herein.

In certain embodiments, the modified cell of leukemic origin is CD34-positive, CD1a-positive, and CD83-positive. In certain embodiments, the modified cell of leukemic origin comprises a cell surface marker selected from the group consisting of CD14, DC-SIGN, Langerin, CD40, CD70, CD80, CD83, CD86, and any combination thereof. In certain embodiments, the modified cell of leukemic origin comprises an MHC class I molecule. In certain embodiments, the modified cell of leukemic origin comprises an MHC class II molecule.

In certain embodiments, the modified cell of leukemic origin comprises a genetic aberration between chromosome 11p15.5 to 11p12. In certain embodiments, the genetic aberration encompasses about 16 Mb of genomic regions (e.g., from about 20.7 Mb to about 36.6 Mb). In certain embodiments, the genetic aberration contains a loss of about 60 known and unknown genes.

In certain embodiments, the modified cell of leukemic origin comprises a co-stimulatory molecule. In certain embodiments, the co-stimulatory molecule includes, without limitation, an MHC class I molecule, BTLA and Toll ligand receptor. Examples of co-stimulatory molecules include CD70, CD80, CD86, 4-1BBL (CD137-ligand), OX4OL, CD30L, CD40, PD-L1, ICOSL, ICAM-1, lymphocyte function-associated antigen 3 (LFA3 (CD58)), K12/SECTM1, LIGHT, HLA-E, B7-H3 and CD83.

In certain embodiments, the modified cell of leukemic origin comprises at least one endogenous antigen. Depending on the leukemic origin of the modified cell, the modified cell of leukemic origin may comprise at least one known endogenous antigen that is specific to the leukemic origin. In certain embodiments, the endogenous antigen is a tumor-associated antigen. In certain embodiments, an endogenous tumor-associated antigen may be selected from the group consisting of WT-1, RHAMM, PRAME, p53, Survivin, and MUC-1.

In certain embodiments, the modified cell of leukemic origin comprises an exogenous antigen or peptide fragments thereof. Such an exogenous antigen may be provided to the modified cell of leukemic origin via various antigen loading strategies. For example, strategies for loading a modified cell of leukemic origin may include, without limitation, the use of synthetic long peptides, mRNA loading, peptide-pulsing, protein-loading, tumor lysate-loading, coculturing with a tumor cell, RNA/DNA transfection or viral transduction. Other strategies for loading a modified cell of leukemic origin are known to those of skill in the art and may be used to load a modified cell of leukemic origin with an exogenous antigen. In general, the modified cell of leukemic origin will process the exogenous antigen via particular molecules, e.g., via MHC I or MHC II. As such, an exogenous antigen comprised by the modified cell of leukemic origin may be an MHC class I antigen or an MHC class II antigen. In certain embodiments, the exogenous antigen is a tumor-associated antigen. For example, in certain embodiments, the modified cell of leukemic origin is loaded with NY-ESO-1 peptide and/or WT-1 peptide, or a tumor-independent antigen such as CMVpp65. In certain embodiments, the exogenous antigen is associated with a disease or disorder, e.g., a non-cancer-associated disease or disorder. It will be appreciated by those of ordinary skill in the art that any tumor-associated antigen or antigen associated with a disease or disorder can be provided to the modified cell of leukemic origin described herein. As such, in certain embodiments, a modified cell of leukemic origin comprises any tumor-associated antigen or antigen associated with a disease or disorder contemplated by those skilled in the art.

In certain embodiments, the exogenous antigen is a non-tumor-associated antigen (i.e., a tumor-independent antigen). In certain embodiments, the modified cell of leukemic origin is loaded with a tumor-independent antigen, i.e., an antigen not associated with a tumor.

For example, suitable tumor-independent antigens include, without limitation, proteins of viral, bacterial, fungal origin; allergens, toxins and venoms, or model antigens of various sources such as chicken egg ovalbumin and keyhole limpet hemocyanin from the giant keyhole limpet, Megathura crenulata. In certain embodiments, a suitable tumor-independent antigen is of bacterial origin. In certain embodiments, a suitable tumor-independent antigen is a diphtheria toxin. In certain embodiments, a suitable tumor-independent antigen is a non-toxic variant of diphtheria toxin. For example, in certain embodiments, a suitable tumor-independent antigen is CRM197 or a variant thereof. In certain embodiments, a modified cell of leukemic origin comprises CRM197 or a variant thereof. In certain embodiments, a suitable tumor-independent antigen is of viral origin. In certain embodiments, a suitable tumor-independent antigen is a peptide derived from cytomegalovirus (CMV), e.g., a peptide derived from CMV internal matrix protein pp65. In certain embodiments, a modified cell of leukemic origin comprises a pp65 peptide. It will be appreciated by those of ordinary skill in the art that any tumor-independent antigen can be provided to the modified cell of leukemic origin described herein. As such, in certain embodiments, a modified cell of leukemic origin comprises any tumor-independent antigen contemplated by those skilled in the art.

In certain embodiments, loading a modified cell of leukemic origin with an exogenous antigen or peptide fragments thereof, includes use of a photochemical processes (e.g., photochemical internalization). In certain embodiments, loading a modified cell of leukemic origin with an exogenous antigen or peptide fragments thereof is achieved with the use of photochemical internalization. In certain embodiments, photochemical internalization may be used to enhance the delivery of an antigen or peptide fragments thereof (e.g., an antigenic polypeptide (e.g., a non-tumor antigen), or a nucleic acid encoding the antigenic polypeptide) into the modified cell of leukemic origin.

Photochemical internalization refers to a delivery method which involves the use of light and a photosensitizing agent for introducing otherwise membrane-impermeable molecules into the cytosol of a target cell, but which does not necessarily result in destruction or death of the target cell. In this method, the molecule to be internalized or transferred is applied to the cells in combination with a photosensitizing agent. Exposure of the cells to light of a suitable wavelength activates the photosensitizing agent which in turn leads to disruption of the intracellular compartment membranes and the subsequent release of the molecule into the cytosol. In photochemical internalization, the interaction between the photosensitizing agent and light is used to affect the cell such that intracellular uptake of the molecule is improved. Photochemical internalization as well as various photosensitizing agents are described in PCT Publication Nos. WO 96/07432, WO 00/54708, WO 01/18636, WO 02/44396, WO 02/44395, and WO 03/020309, U.S. Pat. Nos. 6,680,301, 5,876,989, the disclosures of which are incorporated by reference herein in their entireties. In certain embodiments, photochemical internalization is used to deliver an antigen into the cytosol of a tumor cell. In certain embodiments, photochemical internalization is used to enhance the delivery of an antigen into the cytosol of a tumor cell.

Loading of the modified cell of leukemic origin with an exogenous antigen or peptide fragments thereof may be performed at any time. The skilled person will be able to determine and carry out the specific timing of loading of the modified cell of leukemic origin to best suit their needs. For example, in certain embodiments, the modified cell of leukemic origin is loaded with an exogenous antigen or peptide fragments thereof prior to its exhibiting a mature dendritic cell phenotype. In certain embodiments, the modified cell of leukemic origin is loaded with the exogenous antigen or peptide fragments thereof during transition of the modified cell of leukemic origin to a mature dendritic cell phenotype. In certain embodiments, the modified cell of leukemic origin is loaded with the exogenous antigen or peptide fragments thereof after the modified cell of leukemic origin exhibits a mature dendritic cell phenotype.

In certain embodiments, the modified cell of leukemic origin is a cell of cell line DCOne as described in PCT Publication Nos. WO 2014/006058 and WO 2014/090795, the disclosures of which are incorporated by reference herein in their entireties. In certain embodiments, modified cell of leukemic origin is a cell of cell line DCOne and comprises a mature dendritic cell phenotype that is CD34-positive, CD1a-positive, and CD83-positive. In certain embodiments, modified cell of leukemic origin is a cell of cell line DCOne and is CD34-positive, CD1a-positive, and CD83-positive. In certain embodiments, modified cell of leukemic origin is a cell of cell line DCOne and comprises a cell surface marker selected from the group consisting of CD14, DC-SIGN, Langerin, CD80, CD86, CD40, CD70, and any combination thereof. In certain embodiments, modified cell of leukemic origin is a cell of cell line DCOne and comprises MHC class I. In certain embodiments, modified cell of leukemic origin is a cell of cell line DCOne and comprises MHC class II. In certain embodiments, modified cell of leukemic origin is a cell of cell line DCOne and comprises a genetic aberration between chromosome 11p15.5 to 11p12. In certain embodiments, modified cell of leukemic origin is a cell of cell line DCOne and comprises a genetic aberration that encompasses about 16 Mb of genomic regions (e.g., from about 20.7 Mb to about 36.6 Mb). In certain embodiments, modified cell of leukemic origin is a cell of cell line DCOne and comprises a genetic aberration that contains a loss of about 60 known and unknown genes.

In one aspect, the modified cell of leukemic origin of the present disclosure comprises a downregulated CD47 pathway. CD47 is ubiquitously expressed and functions as a ligand for signal regulatory protein (SIRP)α, which is expressed on myeloid cells, including macrophages and dendritic cells (DCs). CD47 provides a “do not eat me” signal to macrophages through SIRPα to prevent phagocytosis, so that macrophages mediate robust rejection of CD47-deficient cells. See, e.g., Li et al., Nature Comm. (2020) 11: 581, the disclosure of which is incorporated by reference herein in its entirety. As such, as used herein, the “CD47 pathway” refers to the network of molecules that facilitate communication between cells through CD47 and SIRPα. A downregulated CD47 pathway, thus refers to downregulation of any one of the network of molecules that facilitate communication between cells through CD47 and SIRPα.

While current developments in CD47 immunotherapy are directed to blocking CD47 on the surface of cancer cells, the present disclosure relates to CD47 immunotherapy that is directed to downregulating the CD47 pathway on the surface of cells used to treat a cancer. Such downregulation functions to enhance the ability of immune cells to engulf cells that are used to treat the cancer (e.g., a modified cell of leukemic origin based vaccine). Without being bound by any theory, enhancing the uptake of cells used to treat the cancer may result in increased efficacy and biological activity of the cells used to treat the cancer.

In certain embodiments, the downregulated CD47 pathway is the result of the depletion and/or inhibition of a member of the CD47 pathway. In certain embodiments, the member of the CD47 pathway is CD47. As such, provided herein are modified cells of leukemic origin comprising a downregulated CD47 pathway as a result of depletion and/or inhibition of CD47. As described herein, a modified cell of leukemic origin (e.g., DCP-001) comprising a downregulated CD47 pathway, was found to exhibit enhanced uptake by antigen presenting cells. See, Example 2.

In certain embodiments, the downregulated CD47 pathway may be mediated by an agent that depletes and/or inhibits CD47. The agent can be any agent known in the art that functions to deplete and/or inhibit CD47. For example, the agent can be, without limitation, a binding polypeptide (e.g., an antibody), a small molecule, a small RNA, or an engineered nuclease system. In certain embodiments, the small RNA may be a small interfering RNA (siRNA) or a microRNA (miRNA).

In certain embodiments, the down regulated CD47 pathway may be mediated by an agent that modulates CD47 and/or SIRPα (e.g., an anti-CD47 antibody or anti-SIRPα antibody). Various agents that modulate CD47 and/or SIRPα are known to those of ordinary skill in the art and continue to be developed by various companies, and include, for example, Hu5F9-G4 (Forty Seven), TI-061 (Arch Oncology), TTI-662 (Trillium Therapeutics), TTI-621 (Trillium Therapeutics), SRF231 (Surface Oncology), SHR-1603 (Hengrui), OSE-172 (Boehringer Ingelheim), NI-1701 (Novimmune SA), IBI188 (Innovent Biologics), CC-95251 (Celgene), CC-90002 (Celgene), AO-176 (Arch Oncology), ALX148 (ALX Oncology), IMM01 (ImmuneOnco Biopharma), TJC4 (I-MAB Biopharma), TJC4-CK (I-MAB Biopharma), SY102 (Saiyuan), SL-172154 (Shattuck Labs), PSTx-23 (Paradigm Shift Therapeutics), PDL1/CD47 BsAb (Hanmi Pharmaceuticals), NI-1801 (Novimmune SA), MBT-001 (Morphiex), LYN00301 (LynkCell), IMM2504 (ImmuneOnco Biopharma), IMM2502 (ImmuneOnco Biopharma), IMM03 (ImmuneOnco Biopharma), IMC-002 (ImmuneOncia Therapeutics), IBI322 (Innovent Biologics), HMBD-004B (Hummingbird Bioscience), HMBD-004A (Hummingbird Bioscience), HLX24 (Henlius), FSI-189 (Forty Seven), DSP107 (KAHR Medical), CTX-5861 (Compass Therapeutics), BAT6004 (Bio-Thera), AUR-105 (Aurigene), AUR-104 (Aurigene), an anti-CD47 monoclonal antibody developed by Biocad, ABP-500 (Abpro), ABP-160 (Abpro), BH-29xx (Beijing Hanmi). In certain embodiments, the agent is a soluble CD47 receptor, e.g., a soluble SIRPα protein. In certain embodiments, the soluble CD47 receptor is an Fc fusion protein comprising the CD47 binding domain of SIRPα fused to an Fc domain. For example, TTI-621 (Trillium Therapeutics) is a Fc fusion protein comprising the CD47 binding domain of SIRPα fused to an IgG1 Fc domain, and TTI-622 (Trillium Therapeutics) is a Fc fusion protein comprising the CD47 binding domain of SIRPα fused to an IgG4 Fc domain.

In certain embodiments, an agent that modulates CD47 and/or SIRPα is a druggable modifier of CD47. For example, the druggable modifier may be a glutaminyl-peptide cyclotransferase-like protein (QPCTL), the inhibition and/or deletion of which has been shown to disrupt the SIRPα-CD47 interaction and lead to increased phagocytosis of target cells. See, e.g., Logtenberg et al., Nat. Med. (2019) 25(4): 612-619, the disclosure of which is incorporated by reference herein in its entirety. In certain embodiments, the agent that modulates CD47 and/or SIRPα is an inhibitor of QPCTL.

Agents that block the CD47-SIRPα interaction include, without limitation, anti-CD47 antibodies, anti-CD47 nanobodies, engineered SIRPα variants and other fusion proteins, and siRNAs. Use of such agents have been reported in the academic literature. See, e.g., Alvey et al., Curr. Biol. (2017) 27(14): 2065-2077.e6; Weiskopf et al., Science (2013) 341(6141): 88-91; Ma et al., J. Nanobiotechnol. (2020) 18(1): 12; Liu et al., PLoS One (2015) 10(9): e0137345; Sockolosky et al., Proc. Natl. Acad. Sci. USA (2016) 113(19): E2646-2654; Tseng et al., Proc Natl. Acad. Sci. USA (2013); 110(27): 11103-11108; Weiskopf et al., J. Clin. Invest. (2016) 126(7): 2610-2620; and review by Zhang et al. Front. Immunol. (2020) 11:18, the disclosures of which are incorporated by reference herein in their entireties.

Suitable agents that modulate the CD47-SIRPα interaction include those that are described in PCT Publication Nos.: WO2020043188, WO2020036977, WO2020019135, WO2020009725, WO2019241732, WO2019238012, WO2019201236, WO2019185717, WO2019184912, WO2019179366, WO2019157843, WO2019144895, WO2019138367, WO2019108733, WO2019086573, WO2019042470, WO2019042285, WO2019042119, WO2019034895, WO2019027903, WO2018233575, WO2018137705, WO2018095428, WO2018089508, WO2018075960, WO2018075857, WO2017215585, WO2017196793, WO2017194634, WO2017121771, WO2017053423, WO2017049251, WO2017027422, WO2016188449, WO2016141328, WO2016109415, WO2016081423, WO2016024021, WO2016023040, WO2016022971, WO2015191861, WO2014087248, WO2013119714, WO2013109752, WO2012170250, WO2011143624, WO2010070047, WO2009046541, WO2005044857, WO2002092784, WO1999040940, and WO1997027873, the disclosures of which are incorporated by reference herein in their entireties.

A modified cell of leukemic origin comprising a downregulated CD47 pathway of the present disclosure may be pre-coated with an agent that depletes and/or inhibits a member of the CD47 pathway. In certain embodiments, the modified cell of leukemic origin comprising a downregulated CD47 pathway is pre-coated with an agent that depletes and/or inhibits CD47. For example, the modified cell of leukemic origin comprising a downregulated CD47 pathway can be pre-coated with an anti-CD47 antibody. See, e.g., Li et al., Nature Comm. (2020) 11: 581, the disclosure of which is incorporated by reference herein in its entirety.

In certain embodiments, the agent that depletes and/or inhibits a member of the CD47 pathway is an engineered nuclease system.

Various engineered nuclease systems suitable for use in depleting and/or inhibiting a member of the CD47 pathway are known to those of ordinary skill in the art, and for example, includes, a meganuclease system, a zinc finger nuclease (ZFN) system, a transcription activator-like effector nuclease (TALEN) system, and a clustered regularly interspaced short palindromic repeats (CRISPR) system. The engineered nuclease system may mediate an insertion and/or deletion in a gene locus of a member of the CD47 pathway. In certain embodiments, the engineered nuclease system mediates an insertion and/or deletion in the CD47 gene locus. As such, the present disclosure provides a modified cell of leukemic origin comprising an insertion and/or deletion in a CD47 gene locus or a gene locus of a member of the CD47 pathway. It will be readily appreciated by those of ordinary skill in the art that the insertion and/or deletion in a CD47 gene locus or a gene locus of a member of the CD47 pathway results in downregulated expression of CD47 or the member of the CD47 pathway. The insertion and/or deletion may be mediated by, for example, the repair of a double strand break in the CD47 gene locus. In certain embodiments, the repair is via non-homologous end joining (NHEJ) and homology directed repair (HDR).

In certain embodiments, provided herein is a modified cell of leukemic origin comprising an insertion and/or deletion in a CD47 gene locus, wherein the insertion and/or deletion in the CD47 gene locus results in downregulated expression of CD47.

Various CRISPR systems and their uses for gene editing are known to those of ordinary skill in the art. CRISPR RNA sequences and CRISPR-associated (Cas) genes generate catalytic protein-RNA complexes that utilize the incorporated RNA to generate sequence-specific double strand breaks at a complementary DNA sequence. See, e.g., Bhaya et al., Annu. Rev. Genetics (2011) 45: 273-297, the disclosure of which is incorporated by reference herein in its entirety. The Cas9 nuclease from Streptococcus pyogenes (“Cas9”) can be guided to specific sites in the human genome through base-pair complementation between a 20-nucleotide guide region of an engineered single-guide RNA (sgRNA) and a genomic target sequence. See, e.g., Cong et al., Science (2013) 339(6121): 819-823; Mali et al., Science (2013) 339(6121): 823-826; Cho et al. Nat. Biotechnol. (2013) 31(3): 230-232; and Jinek et al., Elife (2013) 2:e00471, the disclosures of which are incorporated by reference herein in their entireties. A catalytically-inactive programmable RNA-dependent DNA-binding protein (dCas9) can be generated by mutating the endonuclease domains within Cas9 which can modulate transcription in bacteria or eukaryotes either directly or through an incorporated effector domain. See, e.g., Qi et al., Cell (2013) 152(5): 1173-1183; Bikard et al., Nucl. Acids Res. (2013) 41(15): 7429-7437; Gilbert et al., Cell (2013) 154(2): 442-451; and Mali et al., Nat. Biotechnol. (2013) 31(9): 833-838, the disclosures of which are incorporated by reference herein in their entireties.

CRISPR systems are RNA-guided nuclease mediated editing systems. RNA-guided nucleases include, without limitation, naturally-occurring Type II CRISPR nucleases such as Cas9, as well as other nucleases derived or obtained therefrom. Exemplary Cas9 nucleases that may be used in the present disclosure include, but are not limited to, S. pyogenes Cas9 (SpCas9), S. aureus Cas9 (SaCas9), N. meningitidis Cas9 (NmCas9), C. jejuni Cas9 (CjCas9), and Geobacillus Cas9 (GeoCas9). In functional terms, RNA-guided nucleases are defined as those nucleases that: (a) interact with (e.g., complex with) a gRNA; and (b) together with the gRNA, associate with, and optionally cleave or modify, a target region of a DNA that includes (i) a sequence complementary to the targeting domain of the gRNA and, optionally, (ii) an additional sequence referred to as a “protospacer adjacent motif,” or “PAM.” RNA-guided nucleases can be defined, in broad terms, by their PAM specificity and cleavage activity, even though variations may exist between individual RNA-guided nucleases that share the same PAM specificity or cleavage activity. The term RNA-guided nuclease should be understood as a generic term, and not limited to any particular type (e.g., Cas9 vs. Cpfl), species (e.g., S. pyogenes vs. S. aureus) or variation (e.g., full-length vs. truncated or split; naturally-occurring PAM specificity vs. engineered PAM specificity).

Various RNA-guided nucleases may require different sequential relationships between PAMs and protospacers. In general, Cas9s recognize PAM sequences that are 5′ of the protospacer as visualized relative to the top or complementary strand. In addition to recognizing specific sequential orientations of PAMs and protospacers, RNA-guided nucleases generally recognize specific PAM sequences. S. aureus Cas9, for example, recognizes a PAM sequence of NNGRRT, wherein the N sequences are immediately 3′ of the region recognized by the gRNA targeting domain. S. pyogenes Cas9 recognizes NGG PAM sequences. It should also be noted that engineered RNA-guided nucleases can have PAM specificities that differ from the PAM specificities of similar nucleases (such as the naturally occurring variant from which an RNA-guided nuclease is derived, or the naturally occurring variant having the greatest amino acid sequence homology to an engineered RNA-guided nuclease). Modified Cas9s that recognize alternate PAM sequences are known in the art. RNA-guided nucleases are also characterized by their DNA cleavage activity: naturally-occurring RNA-guided nucleases typically form double strand breaks (DSBs) in target nucleic acids, but engineered variants have been produced that generate only single strand breaks (SSBs), or that do not cut at all.

Other exemplary Cas9s may be variants of Cas9 with altered activity. These include, for example, a Cas9 nickase (nCas9), a catalytically dead Cas9 (dCas9), a hyper accurate Cas9 (HypaCas9), a high fidelity Cas9 (Cas9-HF), an enhanced specificity Cas9 (eCas9), and an expanded PAM Cas9 (xCas9). See, e.g., Chen et al. Nature (2017) 550(7676): 407-410; Kleinstiver et al. Nature (2016) 529(7587): 490-495; Slaymaker et al. Science (2016) 351(6268): 84-88; and Hu et al. Nature (2018) 556(7699): 57-63, the disclosures of which are incorporated by reference herein in their entireties

Transcription Activator-Like Effector Nucleases (TALENs) are artificial restriction enzymes generated by fusing the TAL effector DNA binding domain to a DNA cleavage domain. These reagents enable efficient, programmable, and specific DNA cleavage and represent powerful tools for genome editing in situ. Transcription activator-like effectors (TALEs) can be quickly engineered to bind practically any DNA sequence. The term TALEN, as used herein, is broad and includes a monomeric TALEN that can cleave double stranded DNA without assistance from another TALEN. The term TALEN is also used to refer to one or both members of a pair of TALENs that are engineered to work together to cleave DNA at the same site. TALENs that work together may be referred to as a left-TALEN and a right-TALEN, which references the handedness of DNA. See, e.g., U.S. patent application Ser. No. 12/965,590; U.S. Ser. No. 13/426,991 (U.S. Pat. No. 8,450,471); U.S. Ser. No. 13/427,040 (U.S. Pat. No. 8,440,431); U.S. Ser. No. 13/427,137 (U.S. Pat. No. 8,440,432); and U.S. Ser. No. 13/738,381, and U.S. Pat. No. 9,393,257, all of which are incorporated by reference herein in their entirety.

TAL effectors are proteins secreted by Xanthomonas bacteria. The DNA binding domain contains a highly conserved 33-34 amino acid sequence with the exception of the 12th and 13th amino acids. These two locations are highly variable (Repeat Variable Di-residue (RVD)) and show a strong correlation with specific nucleotide recognition. This simple relationship between amino acid sequence and DNA recognition has allowed for the engineering of specific DNA binding domains by selecting a combination of repeat segments containing the appropriate RVDs.

The non-specific DNA cleavage domain from the end of the Fok1 endonuclease can be used to construct hybrid nucleases that are active in a yeast assay. These reagents are also active in plant cells and in animal cells. Initial TALEN studies used the wild-type Fok1 cleavage domain, but some subsequent TALEN studies also used Fok1 cleavage domain variants with mutations designed to improve cleavage specificity and cleavage activity. The Fok1 domain functions as a dimer, requiring two constructs with unique DNA binding domains for sites in the target genome with proper orientation and spacing. Both the number of amino acid residues between the TALEN DNA binding domain and the Fok1 cleavage domain and the number of bases between the two individual TALEN binding sites are parameters for achieving high levels of activity. The number of amino acid residues between the TALEN DNA binding domain and the Fok1 cleavage domain may be modified by introduction of a spacer (distinct from the spacer sequence) between the plurality of TAL effector repeat sequences and the Fok1 endonuclease domain. The spacer sequence may be 12 to 30 nucleotides.

The relationship between amino acid sequence and DNA recognition of the TALEN binding domain allows for designable proteins. In this case, artificial gene synthesis is problematic because of improper annealing of the repetitive sequence found in the TALE binding domain. One solution to this is to use a publicly available software program (DNAWorks) to calculate oligonucleotides suitable for assembly in a two-step PCR; oligonucleotide assembly followed by whole gene amplification. A number of modular assembly schemes for generating engineered TALE constructs have also been reported. Both methods offer a systematic approach to engineering DNA binding domains that is conceptually similar to the modular assembly method for generating zinc finger DNA recognition domains.

Once the TALEN genes have been assembled they are inserted into plasmids; the plasmids are then used to transfect the target cell where the gene products are expressed and enter the nucleus to access the genome. TALENs can be used to edit genomes by inducing double-strand breaks (DSB), which cells respond to with repair mechanisms. In this manner, they can be used to correct mutations in the genome which, for example, cause disease.

MegaTALs are fusion proteins that combine homing endonucleases with modular DNA binding domains of TALENs, resulting in improved DNA sequence targeting and increased gene editing efficiencies. N-terminal fusions of TAL anchors can be employed to increase the specificity and activity of a gene-targeted endonuclease, including one or more homing endonucleases such as one or more of the I-HjeMI, I-CpaMI, and I-OnuI homing endonucleases. MegaTALs can be constructed using the Golden Gate assembly strategy described by Cermak et al, Nucl. Acids Res. (2011) 39: e82-e82, the disclosure of which is incorporated by reference herein in its entirety, using, e.g., an RVD plasmid library and destination vector.

Since megaTALs still cut DNA using homing endonuclease cleavage biochemistry, they engage DNA repair pathways in a manner distinct from all other gene editing nucleases. MegaTALs can be designed and predicted according to the procedures in WO 2013/126794 and WO 2014/191525 and can be used in the present methods.

A meganuclease refers to a double-stranded endonuclease having a polynucleotide recognition site of 14-40 base pairs, which can be either monomeric or dimeric. Meganucleases can be designed and predicted according to the procedures in US 2014/0121115, the disclosure of which is incorporated by reference herein in its entirety, can be used in the present methods. Exemplary meganucleases include, but are not limited to, I-Sce I, I-Chu I, I-Dmo I, I-Cre I, I-Csm I, PI-Sce I, PI-Tli I, PI-Mtu I, I-Ceu I, I-Sce II, I-Sce III, HO, PI-Civ I, PI-Ctr I, PI-Aae I, PI-Bsu I, PI-Dha I, PI-Dra I, PI-May I, PI-Mch I, PI-Mfu I, PI-Mfl I, PI-Mga I, PI-Mgo I, PI Min I, PI-Mka I, PI-MIe I, PI-Mma I, PI-Msh I, PI-Msm I, PI-Mth I, PI-Mtu I, PI-Mxe I, PI-Npu I, PI-Pfu I, PI-Rma I, PI-Spb I, PI-Ssp I, PI-Fac I, PI-Mja I, PI-Pho I, PI-Tag I, PI-Thy I, PI-Tko I, and PI-Tsp I; particularly exemplary meganucleases include I-Sce I, I-Chu I, I-Dmo I, I-Cre I, I-Csm I, PI-Sce I, PI-Pfu I, PI-Tli I, PI-Mtu I, and I-Ceu I; particularly exemplary meganucleases include I-Dmo I, I-Cre I, PI-Sce I, and PI-Pfu I.

Zinc finger nucleases (ZFNs) are enzymes having a DNA cleavage domain and a DNA binding zinc finger domain. ZFNs may be made by fusing the nonspecific DNA cleavage domain of an endonuclease with site-specific DNA binding zinc finger domains. Such nucleases are powerful tools for gene editing and can be assembled to induce double strand breaks (DSBs) site-specifically into genomic DNA. ZFNs allow specific gene disruption as during DNA repair, the targeted genes can be disrupted via mutagenic non-homologous end joint (NHEJ) or modified via homologous recombination (HR) if a closely related DNA template is supplied.

Zinc finger proteins can be designed and predicted according to the procedures in WO 98/54311, U.S. Pat. Nos. 9,187,758, 9,206,404 and 8,771,985, the disclosures of which are incorporated by reference herein in their entireties, can be used in the present methods. WO 98/54311, incorporated herein by reference, discloses technology which allows the design of zinc finger protein domains that bind specific nucleotide sequences that are unique to a target gene. It has been calculated that a sequence comprising 18 nucleotides is sufficient to specify a unique location in the genome of higher organisms. Typically, therefore, the zinc finger protein domains are hexadactyl, i.e., contain 6 zinc fingers, each with its specifically designed alpha helix for interaction with a particular triplet. However, in some instances, a shorter or longer nucleotide target sequence may be desirable. Thus, the zinc finger domains in the proteins may contain at least 3 fingers, or from 2-12 fingers, or 3-8 fingers, or 3-4 fingers, or 5-7 fingers, or even 6 fingers. In one aspect, the ZFP contains 3 zinc fingers; in another aspect, the ZFP contains 4 zinc fingers. Additional description on ZFNs and their design for genome editing may be found in US 20120329067, incorporated herein by reference.

Also provided herein are methods for producing a modified cell of leukemic origin comprising a downregulated CD47 pathway. In certain embodiments, such methods comprise incubating a precursor cell under conditions that allow differentiation of the precursor cell into an immature cell; and incubating the immature cell in the presence of an agent that depletes and/or inhibits CD47 and/or a member of the CD47 pathway and under conditions that allows for maturation of the immature cell, thereby producing the modified cell of leukemic origin comprising a downregulated CD47 pathway.

In certain embodiments, the precursor cell is a DCOne cell. As such, provided herein are methods for producing a modified cell of leukemic origin comprising a downregulated CD47 pathway, wherein the method comprises incubating a DCOne cell under conditions that allow differentiation of the DCOne cell into an immature cell (e.g., a cell having an immature dendritic cell phenotype), and incubating the immature cell in the presence of an agent that depletes and/or inhibits CD47 and/or a member of the CD47 pathway and under conditions that allows for maturation of the immature cell (e.g., into a cell having a mature dendritic cell phenotype), thereby producing the modified cell of leukemic origin comprising a downregulated CD47 pathway. The conditions that allow for maturation of DCOne cells, are for instance, described in EP2931878B1, the disclosure of which is incorporated by reference herein in its entirety.

The amount of the agent that depletes and/or inhibits CD47 and/or a member of the CD47 pathway can be readily determined by those of ordinary skill in the art. In certain embodiments, the amount of the agent is an effective amount of the agent to result in the depletion and/or inhibition of CD47 and/or a member of the CD47 pathway.

Where the agent is a biological agent, e.g., a binding polypeptide (e.g., an antibody), a small RNA (e.g., a siRNA or miRNA), or an engineered nuclease system, the modified cell of leukemic origin is generally engineered by introducing one or more engineered nucleic acids encoding the agent or components of the agent.

In certain embodiments, the agent (e.g., antibody, small RNA, or engineered nuclease system) that depletes and/or inhibits CD47 and/or a member of the CD47 pathway is introduced into a cell by an expression vector, e.g., an expression comprising a nucleic acid encoding the agent. Suitable expression vectors are well known to those of ordinary skill in the art and include, without limitation, lentivirus vectors, gamma retrovirus vectors, foamy virus vectors, adeno associated virus (AAV) vectors, adenovirus vectors, engineered hybrid viruses, naked DNA, including but not limited to transposon mediated vectors, such as Sleeping Beauty, Piggybac, and Integrases such as Phi31. Some other suitable expression vectors include Herpes simplex virus (HSV) and retrovirus expression vectors.

In certain embodiments, the nucleic acid encoding the agent (e.g., antibody, small RNA, or engineered nuclease system) that depletes and/or inhibits CD47 and/or a member of the CD47 pathway is introduced into the cell via viral transduction. In certain embodiments, the viral transduction comprises contacting the modified cell of leukemic origin with a viral vector comprising the nucleic acid encoding the agent.

Adenovirus expression vectors are based on adenoviruses, which have a low capacity for integration into genomic DNA but a high efficiency for transfecting host cells. Adenovirus expression vectors contain adenovirus sequences sufficient to: (a) support packaging of the expression vector and (b) to ultimately express the immune receptor in the host cell. In certain embodiments, the adenovirus genome is a 36 kb, linear, double stranded DNA, where a foreign DNA sequence (e.g., a nucleic acid encoding an agent that depletes and/or inhibits CD47 and/or a member of the CD47 pathway) may be inserted to substitute large pieces of adenoviral DNA in order to make the expression vector of the present invention (see, e.g., Danthinne and Imperiale, Gene Therapy (2000) 7(20): 1707-1714, the disclosure of which is incorporated by reference herein in its entirety).

Another expression vector is based on an adeno associated virus (AAV), which takes advantage of the adenovirus coupled systems. This AAV expression vector has a high frequency of integration into the host genome. It can infect nondividing cells, thus making it useful for delivery of genes into mammalian cells, for example, in tissue cultures or in vivo. The AAV vector has a broad host range for infectivity. Details concerning the generation and use of AAV vectors are described in U.S. Pat. Nos. 5,139,941 and 4,797,368, the disclosures of which are incorporated by reference herein in their entireties.

Retrovirus expression vectors are capable of integrating into the host genome, delivering a large amount of foreign genetic material, infecting a broad spectrum of species and cell types and being packaged in special cell lines. The retroviral vector is constructed by inserting a nucleic acid (e.g., a nucleic acid encoding an agent that depletes and/or inhibits CD47 and/or a member of the CD47 pathway) into the viral genome at certain locations to produce a virus that is replication defective. Though the retroviral vectors are able to infect a broad variety of cell types, integration and stable expression of the agent requires the division of host cells.

Lentiviral vectors are derived from lentiviruses, which are complex retroviruses that, in addition to the common retroviral genes gag, pol, and env, contain other genes with regulatory or structural function (see, e.g., U.S. Pat. Nos. 6,013,516 and 5,994,136, the disclosures of which are incorporated by reference herein in their entireties). Some examples of lentiviruses include the human immunodeficiency viruses (HIV-1, HIV-2) and the simian immunodeficiency virus (SIV). Lentiviral vectors have been generated by multiply attenuating the HIV virulence genes, for example, the genes env, vif, vpr, vpu and nef are deleted making the vector biologically safe. Lentiviral vectors are capable of infecting non-dividing cells and can be used for both in vivo and ex vivo gene transfer and expression, e.g., of a nucleic acid encoding an agent that depletes and/or inhibits CD47 and/or a member of the CD47 pathway (see, e.g., U.S. Pat. No. 5,994,136, the disclosure of which is incorporated by reference herein in its entirety).

Expression vectors can be introduced into a cell (e.g., a modified cell of leukemic origin) by any means known to persons skilled in the art. The expression vectors may include viral sequences for transfection, if desired. Alternatively, the expression vectors may be introduced by fusion, electroporation, biolistics, transfection, lipofection, or the like. The cell may be grown and expanded in culture before introduction of the expression vectors, followed by the appropriate treatment for introduction and integration of the vectors. The cells are then expanded and may be screened by virtue of a marker present in the vectors. Various markers that may be used are known in the art, and may include hprt, neomycin resistance, thymidine kinase, hygromycin resistance, etc.

Additional methods for generating a modified cell of leukemic origin comprising a downregulated CD47 pathway include, without limitation, chemical transformation methods (e.g., using calcium phosphate, dendrimers, liposomes and/or cationic polymers), non-chemical transformation methods (e.g., electroporation, optical transformation, gene electrotransfer and/or hydrodynamic delivery) and/or particle-based methods (e.g., impalefection, using a gene gun and/or magnetofection).

Physical methods for introducing an expression vector into cells include calcium phosphate precipitation, lipofection, particle bombardment, microinjection, electroporation, and the like. Methods for producing cells including vectors and/or exogenous nucleic acids are well-known in the art. See, e.g., Sambrook et al. (2001), Molecular Cloning: A Laboratory Manual, Cold Spring Harbor Laboratory, New York. Chemical methods for introducing an expression vector into a host cell include colloidal dispersion systems, such as macromolecule complexes, nanocapsules, microspheres, beads, and lipid-based systems including oil-in-water emulsions, micelles, mixed micelles, and liposomes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform may be used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., Glycobiology (1991) 5: 505-10). Compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the agent, in order to confirm the presence of the nucleic acids in the host cell, a variety of assays may be performed. Such assays include, for example, molecular biology assays well known to those of skill in the art, such as Southern and Northern blotting, RT-PCR and PCR; biochemistry assays, such as detecting the presence or absence of a particular peptide, e.g., by immunological means (ELISAs and Western blots).

In one embodiment, the nucleic acids introduced into the cell are RNA. In another embodiment, the RNA is mRNA that comprises in vitro transcribed RNA or synthetic RNA. The RNA may be produced by in vitro transcription using a polymerase chain reaction (PCR)-generated template. DNA of interest from any source can be directly converted by PCR into a template for in vitro mRNA synthesis using appropriate primers and RNA polymerase. The source of the DNA may be, for example, genomic DNA, plasmid DNA, phage DNA, cDNA, synthetic DNA sequence or any other appropriate source of DNA.

PCR may be used to generate a template for in vitro transcription of mRNA which is then introduced into cells. Methods for performing PCR are well known in the art. Primers for use in PCR are designed to have regions that are substantially complementary to regions of the DNA to be used as a template for the PCR. “Substantially complementary,” as used herein, refers to sequences of nucleotides where a majority or all of the bases in the primer sequence are complementary. Substantially complementary sequences are able to anneal or hybridize with the intended DNA target under annealing conditions used for PCR. The primers can be designed to be substantially complementary to any portion of the DNA template. For example, the primers can be designed to amplify the portion of a gene that is normally transcribed in cells (the open reading frame), including 5′ and 3′ UTRs. The primers may also be designed to amplify a portion of a gene that encodes a particular domain of interest. In one embodiment, the primers are designed to amplify the coding region of a human cDNA, including all or portions of the 5′ and 3′ UTRs. Primers useful for PCR are generated by synthetic methods that are well known in the art. “Forward primers” are primers that contain a region of nucleotides that are substantially complementary to nucleotides on the DNA template that are upstream of the DNA sequence that is to be amplified. “Upstream” is used herein to refer to a location 5, to the DNA sequence to be amplified relative to the coding strand. “Reverse primers” are primers that contain a region of nucleotides that are substantially complementary to a double-stranded DNA template that are downstream of the DNA sequence that is to be amplified. “Downstream” is used herein to refer to a location 3′ to the DNA sequence to be amplified relative to the coding strand.

Chemical structures that have the ability to promote stability and/or translation efficiency of the RNA may also be used. The RNA typically has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of mRNA. Therefore, 3′ UTRs can be selected or designed to increase the stability of the transcribed RNA based on properties of UTRs that are well known in the art.

In one embodiment, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many mRNAs is known in the art. In other embodiments the 5′ UTR can be derived from an RNA virus whose RNA genome is stable in cells. In other embodiments various nucleotide analogues can be used in the 3′ or 5′ UTR to impede exonuclease degradation of the mRNA.

To enable synthesis of RNA from a DNA template without the need for gene cloning, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one embodiment, the promoter is a T7 polymerase promoter, as described elsewhere herein. Other useful promoters include, but are not limited to, T3 and SP6 RNA polymerase promoters. Consensus nucleotide sequences for T7, T3 and SP6 promoters are known in the art.

In one embodiment, the mRNA has both a cap on the 5′ end and a 3′ poly(A) tail which determine ribosome binding, initiation of translation and stability mRNA in the cell. On a circular DNA template, for instance, plasmid DNA, RNA polymerase produces a long concatameric product which is not suitable for expression in eukaryotic cells. The transcription of plasmid DNA linearized at the end of the 3′ UTR results in normal sized mRNA which is not effective in eukaryotic transfection even if it is polyadenylated after transcription.

On a linear DNA template, phage T7 RNA polymerase can extend the 3′ end of the transcript beyond the last base of the template (Schenborn and Mierendorf, Nucl. Acids Res. (1985) 13: 6223-36; Nacheva and Berzal-Herranz, Eur. J. Biochem. (2003) 270: 1485-65.

The polyA/T segment of the transcriptional DNA template can be produced during PCR by using a reverse primer containing a polyT tail, such as 100T tail (size can be 50-5000 T), or after PCR by any other method, including, but not limited to, DNA ligation or in vitro recombination. Poly(A) tails also provide stability to RNAs and reduce their degradation. Generally, the length of a poly(A) tail positively correlates with the stability of the transcribed RNA. In one embodiment, the poly(A) tail is between 100 and 5000 adenosines.

Poly(A) tails of RNAs can be further extended following in vitro transcription with the use of a poly(A) polymerase, such as E. coli polyA polymerase (E-PAP). In one embodiment, increasing the length of a poly(A) tail from 100 nucleotides to between 300 and 400 nucleotides results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps also provide stability to RNA molecules. In an exemplary embodiment, RNAs produced by the methods disclosed herein include a 5′ cap. The 5′ cap is provided using techniques known in the art and described herein (See, e.g., Cougot et al., Trends in Biochem. Sci. (2001) 29: 436-444; Stepinski et al., RNA (2001) 7: 1468-95; Elango et al., Biochim. Biophys. Res. Commun. (2005) 330: 958-966).

In certain embodiments, RNA is electroporated into the cells, such as in vitro transcribed RNA. Any solutes suitable for cell electroporation, which can contain factors facilitating cellular permeability and viability such as sugars, peptides, lipids, proteins, antioxidants, and surfactants can be included.

The methods also provide the ability to control the level of expression over a wide range by changing, for example, the promoter or the amount of input RNA, making it possible to individually regulate the expression level. Furthermore, the PCR-based technique of mRNA production greatly facilitates the design of the mRNAs with different structures and combination of their domains.

One advantage of RNA transfection is that it is essentially transient and a vector-free. An RNA transgene can be delivered to a cell and expressed therein, as a minimal expressing cassette without the need for any additional viral sequences. Under these conditions, integration of the transgene into the cell genome is unlikely. Cloning of cells is not necessary because of the efficiency of transfection of the RNA.

Engineering of cells with in vitro-transcribed RNA (IVT-RNA) includes the use of lipofection or electroporation. It is desirable to stabilize IVT-RNA using various modifications in order to achieve prolonged expression of transferred IVT-RNA.

Some IVT vectors are known in the literature which are utilized in a standardized manner as template for in vitro transcription and which have been genetically modified in such a way that stabilized RNA transcripts are produced. Currently, protocols used in the art are based on a plasmid vector with the following structure: a 5′ RNA polymerase promoter enabling RNA transcription, followed by a gene of interest which is flanked either 3′ and/or 5′ by untranslated regions (UTR), and a 3′ polyadenyl cassette containing 50-70 A nucleotides. Prior to in vitro transcription, the circular plasmid is linearized downstream of the polyadenyl cassette by type II restriction enzymes (recognition sequence corresponds to cleavage site). The polyadenyl cassette thus corresponds to the later poly(A) sequence in the transcript. As a result of this procedure, some nucleotides remain as part of the enzyme cleavage site after linearization and extend or mask the poly(A) sequence at the 3′ end. It is not clear, whether this nonphysiological overhang affects the amount of protein produced intracellularly from such a construct.

In another aspect, the RNA construct is delivered into the cells by electroporation. See, e.g., the formulations and methodology of electroporation of nucleic acid constructs into mammalian cells as taught in US 2004/0014645, US 2005/0052630, US 2005/0070841, US 2004/0059285, US 2004/0092907A1. The various parameters including electric field strength required for electroporation of any known cell type are generally known in the relevant research literature as well as numerous patents and applications in the field. See e.g., U.S. Pat. Nos. 6,678,556, 7,171,264, and 7,173,116. Apparatus for therapeutic application of electroporation are available commercially, e.g., the MEDPULSERTM DNA Electroporation Therapy System (lnovio/Genetronics, San Diego, Calif.), and are described in patents such as U.S. Pat. Nos. 6,567,694; 6,516,223, 5,993,434, 6,181,964, 6,241,701, and 6,233,482; electroporation may also be used for transfection of cells in vitro as described e.g., in US20070128708A1. Electroporation may also be utilized to deliver nucleic acids into cells in vitro. Accordingly, electroporation-mediated administration into cells of nucleic acids including expression constructs utilizing any of the many available devices and electroporation systems known to those of skill in the art presents an exciting new means for delivering an RNA of interest to a target cell.

As provided herein, certain methods utilize the use of a modified cell of leukemic origin, wherein the modified cell is non-proliferating. In certain embodiments, the modified cell of leukemic origin is irradiated. In certain embodiments, the modified cell of leukemic origin is irradiated prior to its use in a method disclosed herein. Irradiation can, for example, be achieved by gamma irradiation at 30-150 Gy, e.g., 100 Gy, for a period of 1 to 3 hours, using a standard irradiation device (Gammacell or equivalent). Irradiation ensures that any remaining progenitor cell in a composition comprising the modified cell of leukemic origin, e.g., a CD34 positive cell, cannot continue dividing. The cells may, for example, be irradiated prior to injection into patients, when used as a vaccine, or immediately after cultivating is stopped. In certain embodiments, the cells are irradiated to inhibit their capacity to proliferate and/or expand, while maintaining their immune stimulatory capacity

### C. METHODS OF TREATMENT

Provided herein are methods for enhancing an immune response in a subject. Also provided are methods for treating or preventing a cancer (e.g., a tumor) in a subject. Methods to enhance an immune response in a subject may result in the treatment of a cancer (e.g., a tumor) that the subject suffers from. The methods generally comprise administering to the subject a modified cell of leukemic origin described herein.

As used herein, the terms “subject” or “individual” or “patient,” are used interchangeably herein, and refers to any subject, particularly a mammalian subject, for whom diagnosis or therapy is desired. Mammalian subjects include for example, humans, domestic animals, farm animals, and zoo, sports, or pet animals such as dogs, cats, guinea pigs, rabbits, rats, mice, horses, cattle, and cows.

As used herein, the terms “treat” or “treatment” refer to both therapeutic treatment and prophylactic or preventative measures, wherein the object is to prevent or slow down (lessen) an undesired physiological change or disorder, such as the development or spread of cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to or at risk of having the condition or disorder or those in which the condition or disorder is to be prevented. In certain embodiments, treatment also refers to preventing recurrence and delaying recurrence of a disease or disorder, e.g., a cancer.

As used herein, an “effective amount” is an amount sufficient to effect beneficial or desired results, e.g., the attainment of a desired therapeutic endpoint (e.g., partial or full reduction in size of a tumor). An effective amount can be administered in one or more administrations, applications or dosages. As used herein, a “therapeutically effective amount” is used to mean an amount sufficient to prevent, correct and/or normalize an abnormal physiological response or a measurable improvement in a desirable response (e.g., enhanced adaptive immune response). In one aspect, a “therapeutically effective amount” is an amount sufficient to reduce by at least about 30%, at least 50% at least 70%, at least 80%, or at least 90%, a clinically significant feature of pathology, such as for example, size of a tumor mass.

Subjects that would benefit from a method of treating a cancer provided herein include those that have cancer. Also suitable are subjects that have previously had an initial treatment for cancer. In certain embodiments, the initial treatment comprises standard of care treatment for the cancer. Standard of care for cancer may include surgery, chemotherapy and/or radiation therapy. Such subjects may have responded well to the initial treatment, or are refractory to the initial treatment. As such, in certain embodiments, methods provided herein are useful for treating a cancer that is refractory to standard of care treatment. In certain embodiments, methods provided herein are useful for treating a subject in order to prevent relapse or recurrence of a cancer.

In certain embodiments, the methods provided by the present disclosure comprise administering to the subject a first composition (e.g., a first immunogenic composition) comprising a modified cell of leukemic origin as described herein. In certain embodiments, the modified cell of leukemic origin comprises a downregulated CD47 pathway. In certain embodiments, the methods provided by the present disclosure comprise administering to the subject a first composition comprising a modified cell of leukemic origin, and a second composition comprising an agent that depletes and/or inhibits CD47 and/or a member of the CD47 pathway. As described herein, inhibiting CD47 enhances the uptake of a cell-based vaccine (e.g., DCP-001) by immune cells. As such, the methods provided herein utilize the immunogenicity of the cell-based vaccine, i.e., to stimulate resident immune cells and/or recruit surrounding immune cells, in combination with enhanced uptake of the cell-based vaccine by such immune cells, to enhance the biological activity of the cell-based vaccine (e.g., by presenting and reacting to antigens comprised by the cell-based vaccine).

In certain embodiments, a method for enhancing an immune response in a subject comprises administering to the subject an effective amount of a composition comprising a modified cell of leukemic origin. In certain embodiments, a method for enhancing an immune response in a subject comprises administering to the subject an effective amount of a composition comprising a modified cell of leukemic origin comprising a downregulated CD47 pathway. In certain embodiments, a method for enhancing an immune response in a subject comprises administering to the subject an effective amount of a first composition comprising a modified cell of leukemic origin, and an effective amount of a second composition comprising an agent that depletes and/or inhibits CD47 and/or a member of the CD47 pathway. In certain embodiments, a method for enhancing an immune response in a subject comprises administering to the subject an effective amount of a first composition comprising a modified cell of leukemic origin comprising a downregulated CD47 pathway, and an effective amount of a second composition comprising an agent that depletes and/or inhibits CD47 and/or a member of the CD47 pathway.

In certain embodiments, a method for treating or preventing a cancer in a subject comprises administering to the subject an effective amount of a composition comprising a modified cell of leukemic origin. In certain embodiments, a method for treating or preventing a cancer in a subject comprises administering to the subject an effective amount of a composition comprising a modified cell of leukemic origin comprising a downregulated CD47 pathway. In certain embodiments, a method for treating or preventing a cancer in a subject comprises administering to the subject an effective amount of a first composition comprising a modified cell of leukemic origin, and an effective amount of a second composition comprising an agent that depletes and/or inhibits CD47 and/or a member of the CD47 pathway. In certain embodiments, a method for treating or preventing a cancer in a subject comprises administering to the subject an effective amount of a first composition comprising a modified cell of leukemic origin comprising a downregulated CD47 pathway, and an effective amount of a second composition comprising an agent that depletes and/or inhibits CD47 and/or a member of the CD47 pathway.

In certain embodiments, the modified cell of leukemic origin comprises at least one tumor associated antigen or a nucleic acid encoding the tumor associated antigen, wherein the tumor associated antigen is associated with the tumor in the subject. In certain embodiments, the modified cell of leukemic origin comprises at least one tumor associated antigen or a nucleic acid encoding the tumor associated antigen, wherein the tumor associated antigen is not associated with the tumor in the subject. In certain embodiments, the tumor associated antigen or a nucleic acid encoding the tumor associated antigen may be comprised by the modified cell of leukemic origin endogenously. In certain embodiments, the tumor associated antigen or a nucleic acid encoding the tumor associated antigen may be provided to the modified cell of leukemic origin exogenously. Various methods for providing a tumor associated antigen or nucleic acid encoding a tumor associated antigen to a cell are known to those of ordinary skill in the art.

In certain embodiments, a method for treating a cancer provided herein comprises administering to a subject one or more doses of an effective amount of a composition comprising a modified cell of leukemic origin described herein (e.g., a modified cell of leukemic origin comprising a downregulated CD47 pathway). The composition may further comprise an agent that depletes and/or inhibits CD47 and/or a member of the CD47 pathway.

In certain embodiments, one or more doses of the composition comprising a modified cell of leukemic origin is administered to the subject. For example, one dose, two doses, three doses, four doses, five doses, six doses, seven doses, eight doses, nine doses, ten doses, eleven doses, twelve doses, or more of the composition comprising a modified cell of leukemic origin is administered to the subject. Each of the one or more doses may contain substantially the same number of modified cells of leukemic origin, or may contain different numbers of modified cells of leukemic origin.

In certain embodiments, doses of the composition (i.e., comprising a modified cell of leukemic origin) may be administered at an interval of time, e.g., at 1 week intervals, at 2 week intervals, at 3 week intervals, at 4 week intervals, at 5 week intervals, at 6 week intervals, at 7 week intervals, at 8 week intervals, at 9 week intervals, at 10 week intervals, at 11 week intervals, at 12 week intervals, or longer. In certain embodiments, the time between doses is from about 1 day to about 21 days, from about 1 day to about 22 days, from about 1 day to about 23 days, from about 1 day to about 24 days, from about 1 day to about 3 weeks, from about 1 day to about 4 weeks, from about 1 day to about 5 weeks, from about 1 day to about 10 weeks, from about 1 day to about 15 weeks, from about 1 day to about 20 weeks, from about 1 day to about 25 weeks, from about 1 day to about 30 weeks, from about 1 day to about 35 weeks, from about 1 day to about 40 weeks, from about 1 day to about 45 weeks, from about 1 day to about 50 weeks, from about 1 day to about 1 year, and any intervening amount of time thereof. In certain embodiments, the time between doses is about 1 day to about 1 month, 14 days to about 2 months, 1 month to about 3 months, 2 months to about 5 months, 4 months to about 6 months, 5 months to about 7 months, 6 months to about 8 months, 7 months to about 9 months, 8 months to about 10 months, 9 months to about 11 months, 10 months to about 12 months, 11 months to about 13 months, 12 months to about 14 months, 13 months to about 15 months, 14 months to about 16 months, 15 months to about 17 months, 16 months to about 18 months, 17 months to about 19 months, 18 months to about 20 months, 19 months to about 21 months, 20 months to about 22 months, 21 months to about 23 months, 22 months to about 24 months, 3 months to about 1 year, 6 months to about 1 year, and any intervening range of time thereof.

As described above, methods described herein include methods comprising the administration of one or more doses of the immunogenic composition. In certain embodiments, the one or more doses are administered via the same route of delivery. In certain embodiments, the one or more doses are administered via different routes of delivery. The methods described herein also include administration of one or more compositions (e.g., a first composition comprising a modified cell of leukemic origin, and a second composition comprising an agent that depletes and/or inhibits CD47 and/or a member of the CD47 pathway).

In certain embodiments, the first and/or second composition is administered intratumorally or peri-tumorally. In such cases, the composition is formulated for intratumoral administration. Intratumoral administration of a composition includes direct administration of the immunogenic composition into a tumor, e.g., into the center of a tumor, or into any location within a tumor mass. Intratumoral administration also includes administration of the composition proximal to a tumor, e.g., the space surrounding the tumor. In certain embodiments, the first and/or the second composition is administered intratumorally. In certain embodiments, the first and/or the second composition is prepared for intratumoral administration, for example, the first and/or the second composition comprises a diluent or solvent acceptable for intratumoral administration.

In certain embodiments, the first and/or second composition is administered extratumorally. In such cases, the composition is formulated for the specific extratumoral administration. Extratumoral administration includes, e.g., parenteral administration, which includes intravenous, intra-arterial, subcutaneous, intradermal, intranodal, intralymphatic and intramuscular administration, which are all well known to the person skilled in the art. In certain embodiments, administration of a composition described herein is delivered by a mode selected from the group consisting of intramuscular injection, subcutaneous injection, intravenous injection, intraarterial injection, intraperitoneal injection, intrasternal injection, intradermal injection, transcutaneous injection, transdermal injection, and delivery to the interstitial space of a tissue.

Extratumoral administration also includes administration to a site distal to a tumor site. For example, extratumoral administration includes administering a composition at a site at least about 0.1 mm, at least about 0.2 mm, at least about 0.3 mm, at least about 0.4 mm, at least about 0.5 mm, at least about 0.6 mm, at least about 0.7 mm, at least about 0.8 mm, at least about 0.9 mm, at least about 1 mm, at least about 2 mm, at least about 3 mm, at least about 4 mm, at least about 5 mm, at least about 6 mm, at least about 7 mm, at least about 8 mm, at least about 9 mm, at least about 10 mm, at least about 15 mm, at least about 20 mm, at least about 25 mm, at least about 30 mm, at least about 35 mm, at least about 40 mm, at least about 45 mm, at least about 50 mm, at least about 60 mm, at least about 70 mm, at least about 80 mm, at least about 90 mm, at least about 10 cm, at least about 20 cm, at least about 30 cm, at least about 40 cm, at least about 50 cm, 50 cm or more away from a tumor (e.g., the edge of a tumor, or the center of a tumor).

Extratumoral administration also includes administering a composition at a site in an organ system that is different to the organ system in which a tumor resides. For example, if the tumor resides at or in an ovary, the method comprises distally administering the composition at a site in an organ system that is not the ovary, e.g., the liver, kidney, etc. The term “organ” or “organ system” as used herein refers to a group of tissues with similar functions. Examples of organ systems include, without limitation, the muscular system, the digestive system (e.g., stomach, small intestine, large intestine, liver, pancreas, etc.), the respiratory system (e.g., lungs), the urinary system (e.g., kidneys, bladder, etc.), the reproductive organs (e.g., male and female reproductive system, ovaries, placenta, prostate, etc.), the endocrine system, the circulatory system, the nervous system (e.g., central and peripheral nervous systems), and the integumentary system (e.g., skin, subcutaneous tissue).

Administration of a composition may also be performed at a site contralateral to the tumor site. In certain embodiments, the method comprises administering a composition at a site contralateral to a tumor site (a site in which the tumor resides). For example, if the tumor resides at or in an ovary, the method comprises distally administering a composition at or in the contralateral ovary. For example, if the tumor resides at or in the left ovary, the method comprises distally administering the composition to the right ovary. For example, if the tumor resides at or in the right ovary, the method comprises distally administering the composition to the left ovary.

In certain embodiments, the first and/or the second composition is administered intravenously. In certain embodiments, the first and/or the second composition is prepared for intravenous administration, for example, the first and/or the second composition comprises a diluent or solvent acceptable for intravenous administration. In certain embodiments, the first and/or the second composition is administered intradermally. In certain embodiments, the first and/or the second composition is prepared for intradermal administration, for example, the first and/or the second composition comprises a diluent or solvent acceptable for intradermal administration. In certain embodiments, the first and/or the second composition is administered intramuscularly. In certain embodiments, the first and/or the second composition is prepared for intramuscular administration, for example, the first and/or the second composition comprises a diluent or solvent acceptable for intramuscular administration.

### D. COMBINATION THERAPY

Methods provided herein are useful in the treatment of cancer by themselves, or in combination with other therapies. As such, also provided herein are combination therapies for use in combination with the methods described herein. For example, methods provided herein can be used in combination with radiation therapy, or with a second therapy having cytostatic or anticancer activity.

In certain embodiments, a method of treating or preventing cancer as described herein further comprises administering to a subject a second therapy. In certain embodiments, the second therapy is comprised within a composition (e.g., a third composition). In certain embodiments, the second therapy comprises radiation therapy. In certain embodiments, the second therapy comprises an immune checkpoint therapy. In certain embodiments, the second therapy comprises an anti-angiogenesis therapy. In certain embodiments, the second therapy comprises a poly (ADP-ribose) polymerase (PARP) inhibitor therapy. Those of skill in the art (e.g., physicians) would readily be able to determine the specific dosages and dosing regimens useful for a combination therapy described herein.

In certain aspects, methods provided herein are useful in combination with a second therapy having cytostatic or anticancer activity. Suitable cytostatic chemotherapy compounds include, but are not limited to DNA cross-linking agents, DNA-fragmenting agents, intercalating agents, protein synthesis inhibitors, topoisomerase I and II inhibitors, antimetabolites, microtubule-directed agents, kinase inhibitors, hormones and hormone antagonists.

In certain aspects, methods provided herein are useful in combination with a second therapy comprising one or more immunooncology (IO) agents. IO agents are known to be effective in enhancing, stimulating, and/or upregulating immune responses in a subject. In certain embodiments, use of an IO agent in combination with a method of treating cancer described herein, results in a synergistic effect in treating the cancer. Examples of IO agents include, without limitation, small molecule drugs, antibodies, and cell-based agents. In certain embodiments, an IO agent is a monoclonal antibody, which can be a human antibody or humanized antibody.

The IO agent can be an agonist of a stimulatory receptor (e.g., a costimulatory receptor), or an antagonist of an inhibitory signal on T cell. The result of both include the amplification of antigen-specific T cell responses. Such IO agents are also referred to in the art as immune checkpoint regulators (e.g., immune checkpoint inhibitors). In certain embodiments, IO agents regulate costimulatory and/or coinhibitory pathways, and are capable of augmenting and/or restoring the function of antigen-specific T cell responses. Examples of molecules involved in costimulatory and/or coinhibitory pathways include, without limitation, members of the immunoglobulin superfamily (IgSF); members of the B7 family of membrane proteins, including, for example, B7-1, B7-2, B7-H1 (PD-L1), B7-DC (PD-L2), B7-H2 (ICOS-L), B7-H3, B7-H4, B7-H5 (VISTA), and B7-H6; members of the tumor necrosis factor (TNF) superfamily, including, for example, CD40, CD4OL, OX-40, OX-40L, CD70, CD27L, CD30, CD3OL, 4-1BBL, CD137 (4-1BB), TRAIL/Apo2-L, TRAILR1/DR4, TRAILR2/DR5, TRAILR3, TRAILR4, OPG, RANK, RANKL, TWEAKR/FnI4, TWEAK, BAFFR, EDAR, XEDAR, TACI, APRIL, BCMA, LTβR, LIGHT, DcR3, HVEM, VEGI/TL1A, TRAMP/DR3, EDAR, EDA1, XEDAR, EDA2, TNFR1, Lymphotoxin α/TNFβ, TNFR2, TNFα, LTβR, Lymphotoxin α1β2, FAS, FASL, RELT, DR6, TROY, and NGFR.

Accordingly, in certain embodiments, the immune checkpoint therapy comprises the use of one or more immune checkpoint regulators that are (i) antagonists of a protein that inhibits T cell activation (e.g., immune checkpoint inhibitors), including, for example, CTLA-4, PD-1, PD-L1, PD-L2, LAG-3, TIM-3, Galectin 9, CEACAM-1, BTLA, CD69, Galectin-1, TIGIT, CD113, GPR56, VISTA, 2B4, CD48, GARP, PD1H, LAIR1, TIM-1, and TIM-4; and (ii) agonists of a protein that stimulates T cell activation, including, for example, B7-1, B7-2, CD28, 4-1BB (CD137), 4-1BBL, ICOS, ICOS-L, OX40, OX40L, GITR, GITRL, CD70, CD27, CD40, DR3 and CD28H.

In certain embodiments, the second therapy as described herein may target one or more immune checkpoint regulators. Immune checkpoint regulators that may be targeted by a second therapy (e.g., an immune checkpoint inhibitor) of the present disclosure may include, without limitation, adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3), V-domain Ig suppressor of T cell activation (VISTA), and NKG2A.

In certain embodiments, a method of treating a cancer as described herein, further comprises administering to the subject an effective amount of an immune checkpoint inhibitor. In certain exemplary embodiments, the immune checkpoint inhibitor targets an immune checkpoint regulator selected from the group consisting of CTLA-4, PD-1, PD-L1,CD47, NKG2A, B7-H3, and B7-H4. In certain embodiments, immune checkpoint inhibitors may be small molecules, recombinant ligands, recombinant receptors, or antibodies. Immune checkpoint inhibitor antibodies may be humanized, human, chimerized, or any form of antibodies known in the art. Accordingly, in certain exemplary embodiments, the immune checkpoint inhibitor is an antibody selected from the group consisting of anti-CTLA-4, anti-PD-1, anti-PD-L1, anti-CD47, anti-NKG2A, anti-B7-H3, and anti-B7-H4. In certain embodiments, the immune checkpoint inhibitor is an antibody selected from the group consisting of ipilimumab, pembrolizumab, nivolumab, atezolizumab, avelumab, durvalumab, and cemiplimab.

In certain embodiments, the immune checkpoint inhibitor is a PD-1 binding antagonist, a molecule that is capable of inhibiting the binding of PD-1 to its ligand binding partners. In certain embodiments, the PD-1 ligand binding partners are PD-L1 and/or PD-L2. In certain embodiments, a PD-L1 binding antagonist is a molecule that inhibits the binding of PDL1 to its binding partners. In certain embodiments, PD-L1 binding partners are PD-1 and/or B7-1. In certain embodiments, the PD-L2 binding antagonist is a molecule that inhibits the binding of PD-L2 to its binding partners. In certain embodiments, a binding partner of PD-L2 is PD-1. Exemplary antibodies are described in U.S. Pat. Nos. 8,735,553, 8,354,509, and 8,008,449, the disclosure of which are incorporated herein by reference in their entireties.

In certain embodiments, the immune checkpoint inhibitor is an anti-PD-1 antibody (e.g., a human antibody, a humanized antibody, or a chimeric antibody). In certain embodiments, the anti-PD-1 antibody is selected from the group consisting of nivolumab, pembrolizumab, and CT-011. Nivolumab, also known as MDX-1106-04, MDX-1106, ONO-4538, BMS-936558, and OPDIVO, is an anti-PD-1 antibody described in International Patent Application No. W02006/121168, the disclosure of which is incorporated herein in its entirety. Pembrolizumab, also known as MK-3475, Merck 3475, lambrolizumab, KEYTRUDA, and SCH-900475, is an anti-PD-1 antibody described in International Patent Application No. WO2009/114335, the disclosure of which is incorporated herein in its entirety. CT-011, also known as Pidilizumab, is an anti-PD-1 antibody described in International Patent Application No. WO2009/101611, the disclosure of which is incorporated herein in its entirety. Additional anti-PD-1 antibodies include PDR001 (Novartis; see WO2015/112900), MEDI-0680 (AMP-514) (AstraZeneca; see WO2012/145493), REGN-2810 (Sanofi/Regeneron; see WO2015/112800), JS001 (Taizhou Junshi), BGB-A317 (Beigene; see WO2015/35606), INCSHR1210 (SHR-1210) (Incyte/Jiangsu Hengrui Medicine; see WO2015/085847), TSR-042 (ANB001) (Tesara/AnaptysBio; see WO2014/179664), GLS-010 (Wuxi/Harbin Gloria Pharmaceuticals), AM-0001 (Armo/Ligand), or STI-1110 (Sorrento; see WO2014/194302), all of which are incorporated by reference herein in their entireties.

In certain embodiments, the immune checkpoint inhibitor is a PD-L1 binding antagonist, such as an antagonistic PD-L1 antibody. Exemplary anti-PD-L1 antibody can be selected from Tecentriq (atezolizumab), durvalumab, avelumab, cemiplimab, STI-1014 (Sorrento; see WO2013/181634), or CX-072 (CytomX; see WO2016/149201). In certain embodiments, the immune checkpoint inhibitor is a PD-L1 antagonist such as Durvalumab, also known as MED14736, atezolizumab, also known as MPDL3280A, or avelumab, also known as MSB00010118C.

In certain embodiments, the immune checkpoint inhibitor is a CTLA-4 binding antagonist, a molecule that is capable of inhibiting the binding of CTLA-4 to its ligand binding partners. CTLA-4 is found on the surface of T cells and acts as an “off” switch when bound to CD80 or CD86, also called B7-1 and B7-2 respectively, on the surface of antigen-presenting cells. CTLA4 is a member of the immunoglobulin superfamily that is expressed on the surface of Helper T cells and transmits an inhibitory signal to T cells. In certain embodiments, the immune checkpoint inhibitor is an anti-CTLA-4 antibody (e.g., a human antibody, a humanized antibody, or a chimeric antibody). Anti-CTLA-4 antibodies are disclosed in U.S. Pat. No. 8,119,129, International Patent Application Nos. WO 01/14424, WO 98/42752; WO 00/37504 (CP675,206, also known as tremelimumab; formerly ticilimumab), U.S. Pat. No. 6,207,156; Hurwitz et al., 1998, the disclosures of which are incorporated herein by reference in their entireties. Antibodies that compete with any of these art-recognized antibodies for binding to CTLA-4 also can be used, for example, a humanized CTLA-4 antibody is described in International Patent Application Nos. WO2001014424, WO2000037504, and U.S. Pat. No. 8,017,114, the disclosures of which are incorporated herein by reference in their entireties. Exemplary anti-CTLA-4 antibodies include, ipilimumab (also known as 10D1, MDX-010, MDX-101, and Yervoy).

In certain embodiments, the immune checkpoint inhibitor is an antibody to B7-H4 (e.g., those disclosed in International Patent Application Nos. WO 2013025779 and WO2013067492, the disclosures of which are incorporated by reference herein in their entireties). In certain embodiments, the immune checkpoint inhibitor is an antibody to B7-H3, including without limitation antibodies neutralizing human B7-H3 (e.g., MGA271 disclosed as BRCA84D and derivatives in U.S. Patent Publication No. 20120294796, the disclosure of which is incorporated by reference herein in its entirety). In certain embodiments, the immune checkpoint inhibitor is an antibody to NKG2A, see, e.g., Montfoort et al. Cell (2018) 175(7):1744-1755, the disclosure of which is incorporated by reference herein in its entirety.

In certain embodiments, the immune checkpoint inhibitor is a macrophage checkpoint blockade. For example, CD47 has been identified as a dominant macrophage checkpoint, and is found to be overexpressed in myeloid malignancies that leads to tumor evasion of phagocytosis by macrophages. CD47 blockade has been shown to result in the engulfment of leukemic cells, and pre-clinical data has shown anti-cancer activity in multiple hematologic malignancies including AML and myelodysplastic syndrome (MDS). See, e.g., Chao et al. Frontiers in Oncology (2019) 9:1380. Accordingly, in certain embodiments, the immune checkpoint inhibitor is an antibody to CD47.

In certain aspects, methods provided herein are useful in combination with a second therapy comprising one or more anti-angiogenic agents. Accordingly, methods provided herein are useful in combination with anti-angiogenesis therapy. The formation of new blood vessels, or angiogenesis, facilitates cancer growth and metastasis by providing a tumor with dedicated blood supply to provide oxygen and essential nutrients required for its growth. Therapies targeting angiogenesis and associated growth factors including, without limitation, vascular endothelial growth factor (VEGF), platelet-derived growth factor (PDGF), and fibroblast growth factor (FGF), have been shown to inhibit new blood vessel growth.

Many anti-angiogenic agents are known in the art and would be suitable for use in combination with a method provided herein. Exemplary anti-angiogenic agents include, without limitation, physiological agents such as growth factors (e.g., ANG-2, NK1, 2, 4 (HGF), transforming growth factor beta (TGF-β)), cytokines (e.g., interferons such as IFN-α, -β, -γ, platelet factor 4 (PF-4), PR-39), proteases (e.g., cleaved AT-III, collagen XVIII fragment (Endostatin)), HmwKallikrein-d5 plasmin fragment (Angiostatin), prothrombin-F1-2, TSP-1), protease inhibitors (e.g., tissue inhibitor of metalloproteases such as TIMP-1, -2, or -3; maspin; plasminogen activator-inhibitors such as PAI-1; pigment epithelium derived factor (PEDF)), Tumstatin, antibody products (e.g., the collagen-binding antibodies HUIV26, HU177, XL313; anti-VEGF: anti-integrin (e.g., Vitaxin, (Lxsys)), and glycosidases (e.g., heparinase-I or -II). Also suitable are molecules that are antagonists to angiogenesis-associated antigens (including proteins and polypeptides), including, without limitation, molecules directed against VEGF, VEGF receptor, EGFR, bFGF, PDGF-B, PD-ECGF, TGFs including TGF-α, endoglin, Id proteins, various proteases, nitric oxide synthase, aminopeptidase, thrombospondins, k-ras, Wnt, cyclin-dependent kinases, microtubules, heat shock proteins, heparin-binding factors, synthases, collagen receptors, integrins, and surface proteoglycan NG2. “Chemical” or modified physiological agents known or believed to have anti-angiogenic potential include, for example, vinblastine, taxol, ketoconazole, thalidomide, dolestatin, combrestatin A, rapamycin (Cuba, et al. Nature Medicine (2002) 8:128-135, the disclosure of which is incorporated by reference herein in its entirety), CEP-7055 (available from Cephalon, Inc.), flavone acetic acid, Bay 12-9566 (Bayer Corp.), AG3340 (Agouron, Inc.). CGS. 27023A (Novartis), tetracylcine derivatives (e.g., COL-3 (Collagenix, Inc.)), Neovastat (Aeterna), BMS-275291 (Bristol-Myers Squibb), low dose 5-FU, low dose methotrexate (MTX), irsofladine, radicicol, cyclosporine, captopril, celecoxib, D45152-sulphated polysaccharide, cationic protein (Protarnine), cationic peptide-VEGF, Suramin (polysulphonated napthyl urea), compounds that interfere with the function or production of VEGF (e.g., SU5416 or SU6668 (Sugen), PTK787/ZK22584 (Novartis)), Distamycin A, Angiozyme (ribozyme), isoflavinoids, staurosporine derivatives, genistein, EMD121974 (Merck KcgaA), tyrphostins, isoquinolones, retinoic acid, carboxyamidotriazole, TNP-470, octreotide, 2-methoxyestradiol, aminosterols (e.g., squalamine), glutathione analogues (e.g., N-acteyl-L-cysteine), combretastatin A-4 (Oxigene), Eph receptor blocking agents (Himanen et al. Nature (2001) 414(6866):933-938, the disclosure of which is incorporated by reference herein in its entirety), Rh-Angiostatin, Rh-Endostatin (see, International Patent Application No. WO 01/93897, the disclosure of which is incorporated by reference herein in its entirety), cyclic-RGD peptide, accutin-disintegrin, benzodiazepenes, humanized anti-avb3 Ab, Rh-PAI-2, amiloride, p-amidobenzamidine, anti-uPA ab, anti-uPAR Ab, L-phenylalanine-N-methylamides (e.g., Batimistat, Marimastat), AG3340, and minocycline.

In certain embodiments, the anti-angiogenesis agent is an anti-VEGF antibody. Exemplary anti-VEGF antibodies include any antibodies, or antigen binding fragments thereof, that bind with sufficient affinity and specificity to VEGF and can reduce or inhibit the biological activity of VEGF. In certain embodiments, anti-VEGF antibodies include, without limitation, a monoclonal antibody that binds to the same epitope as the monoclonal anti-VEGF antibody A4.6.1 produced by hybridoma ATCC HB 10709; a recombinant humanized anti-VEGF monoclonal antibody generated according to Presta et al. Cancer Research (1997) 57:4593-4599, the disclosure of which is incorporated by reference herein in its entirety. In certain embodiments, the anti-VEGF antibody is Bevacizumab (BV), also known as rhuMAb VEGF or AVASTIN. Bevacizumab and other humanized anti-VEGF antibodies are further described in U.S. Pat. No. 6,884,879, the disclosure of which is incorporated by reference herein in its entirety. Additional antibodies include, e.g., G6-31 and B20-4.1, as described in International Patent Application Nos. WO2005/012359 and WO2005/044853, the disclosures of which are incorporated by reference herein in their entireties. Additional anti-VEGF antibodies are described in the following U.S. Pat. Nos. 7,060,269, 6,582,959, 6,703,020, and 6,054,297; International Patent Publication Nos. WO98/45332, WO 96/30046, and WO94/10202; European Patent No. EP 0666868B1; U.S. Patent Publication Nos. 2006009360, 20050186208, 20030206899, 20030190317, 20030203409, and 20050112126; and Popkov et al., Journal of Immunological Methods 288:149-164 (2004), the disclosures of which are incorporated by reference herein in their entireties. Additional VEGF inhibitors include Sunitinib (SUTENT®, Pfizer) and sorafenib (NEXAVAR®, Onyx and Bayer Healthcare Pharmaceuticals) which belong to a group of VEGF-receptor tyrosine-kinase inhibitors (RTKIs) with activity against both VEGFR and PDGFR. In certain embodiments, the anti-angiogenesis agent is sunitinib. Yet other VEGF inhibitors include fusion proteins that prevent ligand binding to vascular endothelial growth factor receptors (VEGFR). These fusion proteins are sometimes referred to as VEGF traps, and include aflibercept. Accordingly, in certain embodiments, the anti-angiogenesis therapy comprises an anti-angiogenesis agent selected from the group consisting of bevacizumab, aflibercept, sunitinib, and sorafenib.

In certain aspects, methods provided herein are useful in combination with a second therapy comprising one or more poly (ADP-ribose) polymerase (PARP) inhibitors. Accordingly, methods provided herein are useful in combination with PARP inhibitor therapy. PARP is a family of proteins involved in many functions in a cell, including DNA repair, gene expression, cell cycle control, intracellular trafficking and energy metabolism. PARP proteins play key roles in single strand break repair through the base excision repair pathway. PARP inhibitors have shown activity as a monotherapy against tumors with existing DNA repair defects, such as BRCA1 and BRCA2, and as a combination therapy when administered together with anti-cancer agents that induce DNA damage. The PARP inhibitor may be selected from the group consisting of a small molecule, a nucleic acid, a nucleic acid analog or derivative, a peptide, a peptidomimetic, a protein, an antibody or an antigen-binding fragment thereof, a monosaccharide, a disaccharide, a trisaccharide, an oligosaccharide, a polysaccharide, a lipid, a glycosaminoglycan, an extract made from a biological material, and combinations thereof. Exemplary PARP inhibitors include, without limitation, olaparib, veliparib or a prodrug thereof, rucaparib, talazoparib, niraparib, INO-1001, AZD2461, SC10914, BGB-290, and Fluzoparib. Accordingly, in certain embodiments, the PARP inhibitor therapy comprises a PARP inhibitor selected from the group consisting of olaparib, niraparib, rucaparib, and veliparib.

Combination therapies described herein comprising a method useful in the treatment of a cancer (e.g., cancer therapy) and a second therapy (e.g., immune checkpoint therapy, anti-angiogenesis therapy, PARP inhibitor therapy) encompass treatment regimens wherein the cancer therapy and the second therapy are simultaneously (e.g., substantially simultaneously) or sequentially administered to a subject. For example, a cancer therapy described herein can be substantially simultaneously administered to a subject together with the second therapy. Substantially simultaneous administration can be accomplished, for example, by administering to the subject a single dosage form having a fixed ratio of each therapy or in multiple, single dosage forms for each therapy. Each therapy can be sequentially or substantially simultaneously administered by any appropriate route including, without limitation, oral routes, intravenous routes, intratumoral routes, intramuscular routes, and direct absorption through mucous membrane tissues.

In certain embodiments, the cancer therapy and the second therapy are administered by the same route or by different routes. For example, a cancer therapy of the combination selected may be administered by intravenous injection while the second therapy of the combination may be administered intratumorally. Alternatively, for example, all therapies may be administered intravenously or all therapeutic agents may be administered by intratumorally.

In certain embodiments, a combination therapy can include the administration of the cancer therapy and the second therapy, in combination with other biologically active ingredients and non-drug therapies (e.g., surgery or radiation treatment). Where the combination therapy further comprises a non-drug treatment, the non-drug treatment may be conducted at any suitable time so long as a beneficial effect from the co-action of the combination of the therapies and non-drug treatment is achieved.

### E. PHARMACEUTICAL COMPOSITIONS AND FORMULATIONS

Also provided are immunogenic compositions comprising a modified cell of leukemic origin (e.g., a modified cell of leukemic origin comprising a downregulated CD47 pathway) of the present disclosure, including pharmaceutical compositions and formulations, such as unit dose form compositions. The pharmaceutical compositions and formulations generally include one or more optional pharmaceutically acceptable carrier or excipient. Therapies of the present disclosure can be constituted in a composition, e.g., a pharmaceutical composition (e.g., an immunogenic pharmaceutical composition) containing a modified cell of leukemic origin (e.g., a modified cell of leukemic origin comprising a downregulated CD47 pathway) and optionally a pharmaceutically acceptable carrier.

In certain embodiments, the composition comprises a modified cell of leukemic origin of the present disclosure (e.g., a modified cell of leukemic origin comprising a downregulated CD47 pathway). In certain embodiments, the composition further comprises an agent that depletes and/or inhibits CD47 and/or a member of the CD47 pathway (e.g., a binding polypeptide, a small molecule, a small RNA, or an engineered nuclease system), as described herein. The agent that depletes and/or inhibits CD47 and/or a member of the CD47 pathway can be co-formulated into the composition at an effective amount to result in depletion and/or inhibition of CD47 and/or a member of the CD47 pathway comprised by the modified cell of leukemic-origin.

A modified cell of leukemic origin comprising a downregulated CD47 pathway of the present disclosure may be pre-coated with an agent that depletes and/or inhibits a member of the CD47 pathway. In certain embodiments, the modified cell of leukemic origin comprising a downregulated CD47 pathway is pre-coated with an agent that depletes and/or inhibits CD47. For example, the modified cell of leukemic origin comprising a downregulated CD47 pathway can be pre-coated with an anti-CD47 antibody. See, e.g., Li et al., Nature Comm. (2020) 11: 581. In certain embodiments, the composition further comprises an anti-CD47 antibody, for example, that coats the modified cell of leukemic origin. As such, the present disclosure provides compositions (e.g., immunogenic compositions) comprising a modified cell of leukemic origin and an anti-CD47 antibody. The present disclosure also provides compositions (e.g., immunogenic compositions) comprising a modified cell of leukemic origin comprising a downregulated CD47 pathway and an anti-CD47 antibody.

In certain embodiments, the composition includes at least one additional therapeutic agent (e.g., a second therapy having cytostatic or anticancer activity).

The term “pharmaceutical formulation” refers to a preparation which is in such form as to permit the biological activity of an active ingredient contained therein to be effective, and which contains no additional components which are unacceptably toxic to a subject to which the formulation would be administered. A “pharmaceutically acceptable carrier” refers to an ingredient in a pharmaceutical formulation, other than an active ingredient, which is nontoxic to a subject. Accordingly, there are a variety of suitable formulations. A pharmaceutically acceptable carrier includes, but is not limited to, a buffer, excipient, stabilizer, or preservative.

In certain embodiments, the choice of carrier is determined in part by the particular cell and/or by the method of administration. A pharmaceutically acceptable carrier includes any and all solvents, dispersion media, coatings, antibacterial and antifungal agents, isotonic and absorption delaying agents, and the like that are physiologically compatible. In certain embodiments, the carrier for a composition containing a modified cell of leukemic origin (e.g., a modified cell of leukemic origin comprising a downregulated CD47 pathway) is suitable for intravenous, intramuscular, subcutaneous, parenteral, spinal or epidermal administration (e.g., by injection or infusion). In certain embodiments, where suitable, e.g., a small molecule based second therapy, the carrier for a composition containing the second therapy is suitable for non-parenteral, e.g., oral administration. A pharmaceutical composition of the disclosure can include one or more pharmaceutically acceptable salts, anti-oxidant, aqueous and non-aqueous carriers, and/or adjuvants such as preservatives, wetting agents, emulsifying agents and dispersing agents. In certain embodiments, the pharmaceutical composition can contain preservatives. Suitable preservatives may include, for example, methylparaben, propylparaben, sodium benzoate, and benzalkonium chloride. In certain embodiments, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to:

buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in certain embodiments are included in the compositions. Suitable buffering agents include, for example, citric acid, sodium citrate, phosphoric acid, potassium phosphate, and various other acids and salts. In certain embodiments, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being treated with the cells, such as those with activities complementary to the cells, where the respective activities do not adversely affect one another. Such active ingredients are suitably present in combination in amounts that are effective for the purpose intended. Thus, in certain embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine. The pharmaceutical composition in certain embodiments contains the cells in amounts effective to treat or prevent the disease or condition, such as a therapeutically effective or prophylactically effective amount. Therapeutic or prophylactic efficacy in certain embodiments is monitored by periodic assessment of treated subjects. The desired dosage can be delivered by a single bolus administration of the cells, by multiple bolus administrations of the cells, or by continuous infusion administration of the cells.

Formulations include those for oral, intravenous, intraperitoneal, subcutaneous, pulmonary, transdermal, intramuscular, intranasal, buccal, sublingual, or suppository administration. In certain embodiments, the cell populations are administered parenterally. The term “parenteral,” as used herein, includes intravenous, intramuscular, subcutaneous, rectal, vaginal, and intraperitoneal administration. In certain embodiments, the cells are administered to the subject using peripheral systemic delivery by intravenous, intraperitoneal, or subcutaneous injection.

In certain embodiments, a method for treating a cancer comprises administering an immunogenic composition comprising a modified cell of leukemic origin (e.g., a modified cell of leukemic origin comprising a downregulated CD47 pathway), wherein the immunogenic composition further comprises a pharmaceutically acceptable carrier. In certain embodiments, the immunogenic composition is formulated for intradermal administration. In certain embodiments, the administration of the immunogenic composition is intradermal. In certain embodiments, the immunogenic composition is formulated for intraperitoneal administration. In certain embodiments, the administration of the immunogenic composition is intraperitoneal. In certain embodiments, the immunogenic composition is formulated for intratumoral administration. In certain embodiments, the administration of the immunogenic composition is intratumoral.

In certain embodiments, the immunogenic composition is formulated for loco-regional lymph node administration. In certain embodiments, the administration of the immunogenic composition is into a loco-regional lymph node. In certain embodiments, loco-regional lymph node administration is performed during or following an initial treatment of the ovarian cancer. In certain embodiments, loco-regional lymph node administration is performed during or following an initial treatment of the ovarian cancer, wherein the initial treatment comprises surgery.

Compositions in certain embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the cells in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts may in some aspects be consulted to prepare suitable preparations. The formulations to be used for in vivo administration are generally sterile. Sterility may be readily accomplished, e.g., by filtration through sterile filtration membranes.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

Actual dosage levels of the active ingredients in the pharmaceutical compositions of the present disclosure can be varied so as to obtain an amount of the active ingredient which is effective to achieve the desired therapeutic response for a particular patient, composition, and mode of administration, without being unduly toxic to the patient. The selected dosage level will depend upon a variety of pharmacokinetic factors including the activity of the particular compositions of the present disclosure employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. A composition of the present disclosure can be administered via one or more routes of administration using one or more of a variety of methods well known in the art. As will be appreciated by the skilled artisan, the route and/or mode of administration will vary depending upon the desired results.

In certain embodiments, the compositions include a cryopreservation agent (CPA). Methods for cryopreservation of cells are well known in the art. See, e.g., C. B. Morris, “Cryopreservaton of Animal and Human Cell Lines” (2007), in Methods in Molecular Biology, vol 368: Cryopreservation and Freeze-Drying Protocols, 2nd Ed. (J. G. Day and G. N. Stacey eds.), Humana Press Inc. Totowa, N.J., pp. 227-236, which is incorporated herein in its entirety. In certain embodiments, compositions comprising a CPA allows for the use of very low temperatures to preserve structural aspects of materials contained within the composition (e.g., a modified cell of leukemic origin described herein). Generally, cryopreservation and use of a CPA allows for the solidification of the composition in a noncrystalline phase. The CPA, which is usually a fluid, reduces the freezing injury from the cryopreservation process. CPAs can be divided into two categories: (1) cell membrane-permeating cryoprotectants, such as dimethyl sulfoxide (DMSO), glycerol, and 1,2-propanediol; and (2) nonmembrane-permeating cryoprotectants, such as 2-methyl-2,4-pentanediol and polymers such as polyvinyl pyrrolidone, hydroxyethyl starch, and various sugars. Suitable CPAs include, without limitation, the CELLBANKER® series of CPAs, the CRYOSTOR® series of CPAs, dimethylsulfoxide (DMSO), ethylene glycol, glycerol, trehalose, propylene glycol, and the like. Further, biomaterials such as alginates, polyvinyl alcohol, and chitosan can be used to impede ice crystal growth, along with traditional small molecules.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

While the present invention has been described with reference to the specific embodiments thereof, it should be understood by those skilled in the art that various changes may be made and equivalents may be substituted without departing from the true spirit and scope of the invention. It will be readily apparent to those skilled in the art that other suitable modifications and adaptations of the methods described herein may be made using suitable equivalents without departing from the scope of the embodiments disclosed herein. In addition, many modifications may be made to adapt a particular situation, material, composition of matter, process, process step or steps, to the objective, spirit and scope of the present invention. All such modifications are intended to be within the scope of the claims appended hereto. Having now described certain embodiments in detail, the same will be more clearly understood by reference to the following examples, which are included for purposes of illustration only and are not intended to be limiting.

### F. EXPERIMENTAL EXAMPLES

### Example 1: Processing of DCP-001 by Antigen Presenting Cells

DCP-001 is an allogeneic cell-based vaccine comprising modified cells of leukemic origin having a mature dendritic cell (DC) phenotype generated through differentiation and maturation of the cell line DCOne. DCOne is deposited under the conditions of the Budapest treaty with the DSMZ under accession number DSMZ ACC3189 on 15 Nov. 2012. The process of obtaining mature cells from the deposited DCOne cell line is for instance described in EP2931878B1, the disclosure of which is incorporated by reference herein in its entirety.

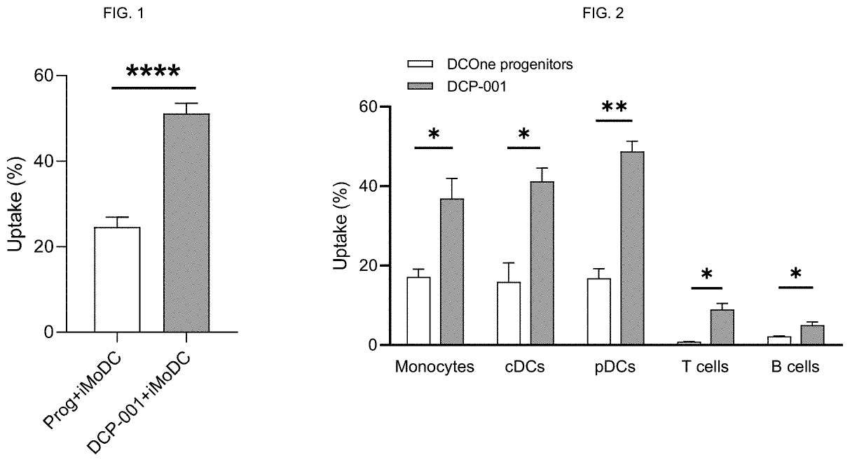

DCP-001 was found to be endocytosed by immature monocyte derived dendritic cells (iMoDCs). The VPD450 dye was used to label iMoDCs and CFSE dye was used to label DCP-001 or DCOne progenitors. VPD450-labeled iMoDCs were cocultured with CFSE-labelled DCP-001 or DCOne progenitors (1:1 ratio) for 4 hours at 37° C. Cells were then stained for antigen presenting cell (APC)-conjugated anti-CD274 for 30 minutes at 4° C. FIG. 1 shows the percentage uptake of DCP-001 or DCOne progenitors by iMoDCs, which was determined as the VPD450/CFSE positive population in the total APC-positive iMoDC population. In FIG. 1, the data represents 16-25 independent experiments; **** indicates a statistically significant difference as calculated by unpaired t-test, with a p<0.0001.

DCP-001 was also found to be taken up by antigen presenting cells in peripheral blood. Peripheral blood mononuclear cells (PBMCs) were co-cultured with CFSE-labelled DCP-001 or DCOne progenitors at a 1:1 ratio for 4 hours. After 4 hours of co-culture, cells were identified as monocytes (CD14⁺), myeloid DCs (CD11c^{hi}, HLA-DR⁺, CD14⁻), plasmacytoid DCs (CD304⁺, HLA-DR⁺), T cells (CD3⁺), B cells (CD19⁺). To quantify uptake, the percentages of the VPD450/CFSE-positive populations in the specific subpopulations of PBMCs were determined. FIG. 2 shows the percentage uptake of DCP-001 or DCOne progenitors by each specific subpopulation of PBMCs, as indicated. In FIG. 2, the data represents 3 independent experiments and is expressed as mean ±SEM; * indicates a statistically significant difference as calculated by unpaired t-test using the Holm-Sidak method, with p<0.05; and ** p<0.01.

### Example 2: Role of Phosphatidylserine (PS) and CD47 in the Processing of DCP-001

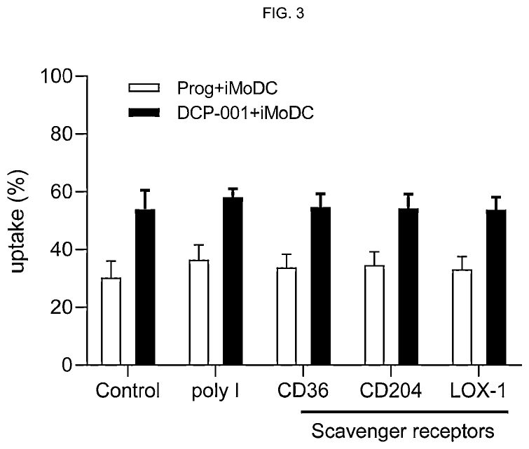

To elucidate the pathways involved in the processing of DCP-001 by antigen presenting cells, the role of scavenger receptors in the uptake of DCP-001 or DCOne progenitors (prog) by host iMoDCs was investigated. It was found that scavenger receptors were redundant in the uptake of DCP-001 by iMoDCs. An agent selected from polyinosinic acid (polyl; 50 μg/mL), anti-CD36 antibody (50 μg/mL), anti-CD204 antibody (50 μg/mL), or anti-LOX-1 antibody (50 μg/mL) was added during the iMoDC uptake assay as described in Example 1. FIG. 3 shows the percentage uptake of DCP-001 or DCOne progenitors by iMoDCs in the presence of each agent. In FIG. 3, the data represents 3 independent experiments and is expressed as mean ±SEM.

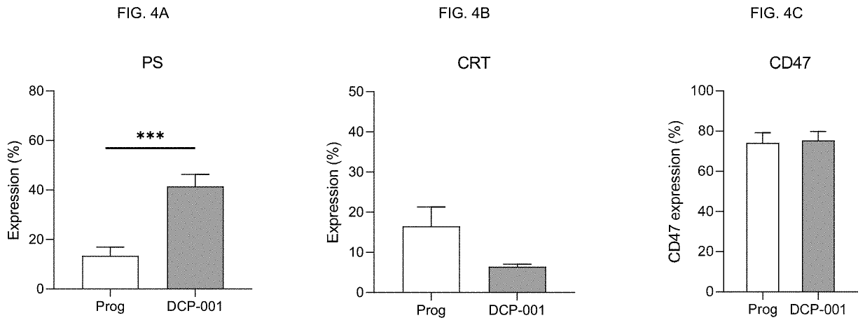

The expression of phosphatidylserine (PS; FIG. 4A), calreticulin (CRT; FIG. 4B), and CD47 (FIG. 4C) on the surface of DCP-001 and DCOne progenitors was determined by flow cytometry. It was found that DCP-001 expresses PS, CRT, and CD47. In FIG. 4A-FIG. 4C, the data represents values obtained from 4-6 batches of DCP-001 and DCOne progenitors, and is expressed as mean ±SEM. In FIG. 4A, *** indicates a statistically significant difference as calculated by unpaired t-test, with p<0.001.

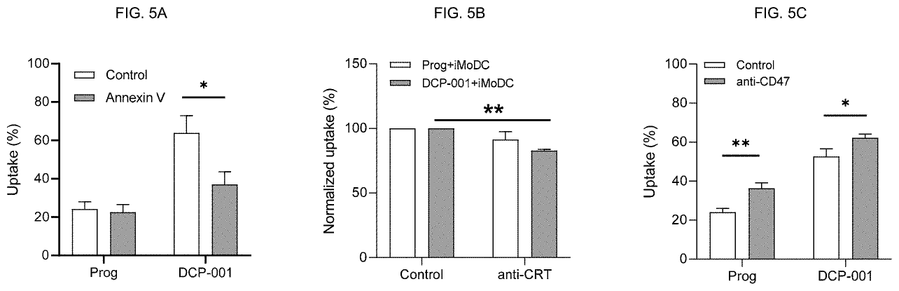

To evaluate the role of PS and CRT (the “eat me” signals) on the uptake of DCP-001 by antigen presenting cells, purified recombinant Annexin V or a CRT-specific antibody was added to iMoDC co-cultured with DCP-001 or DCOne progenitors (FIG. 5A and FIG. 5B, respectively). As shown in FIG. 5A, addition of Annexin V to the co-culture resulted in a significant reduction in the percentage of uptake of DCP-001. In FIG. 5A, the data represents 3-4 independent experiments and is expressed as mean ±SEM; * indicates a statistically significant difference as calculated by paired t-test, with p<0.05. As shown in FIG. 5B, addition of a CRT-specific antibody to the co-culture resulted in significant reduction in the normalized percentage of uptake of DCP-001. Normalized percentage of uptake was calculated by normalizing uptake in the presence of the CRT-specific antibody to control (in the absence of antibody, set at 100%). In FIG. 5B, the data represents 3-4 independent experiments and is expressed as mean±SEM; ** indicates a statistically significant difference as calculated by paired t-test, with p<0.01.

To evaluate the role of the “do not eat me” signal CD47 in the uptake of DCP-001 by antigen presenting cells, a monoclonal antibody targeting CD47 was added to iMoDC co-cultured with DCP-001 or DCOne progenitors (FIG. 5B). As shown in FIG. 5B, blockade of CD47 resulted in enhanced uptake of both DCP-001 and DCOne progenitors. In FIG. 5B, the data represents 3-4 independent experiments and is expressed as mean±SEM; * indicates a statistically significant difference as calculated by paired t test, with p<0.05; and **p<0.005.

### Example 3: Immunogenicity of DCP-001

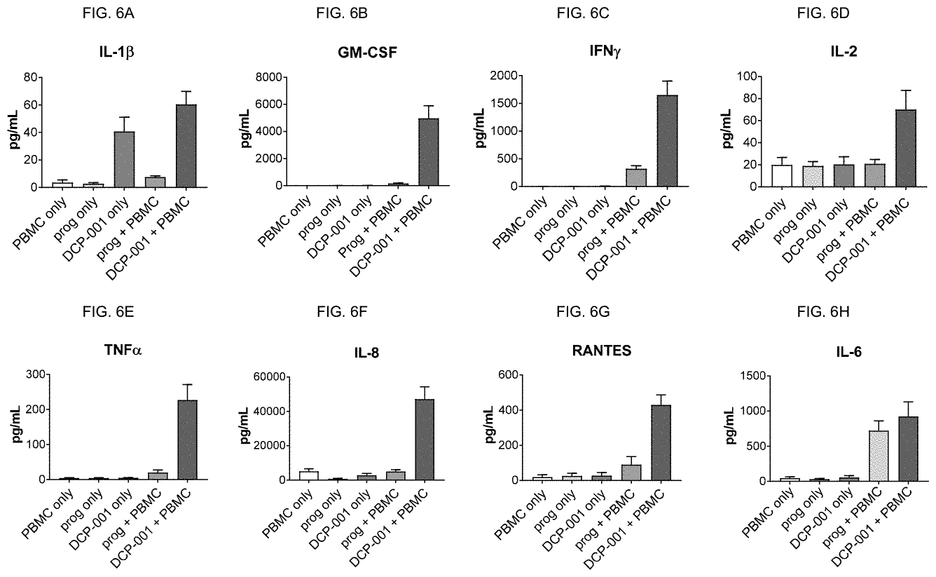

DCP-001 and DCOne progenitors (prog) were tested in a PBMC stimulation assay by co-culture for 6 days after which supernatants were collected for multiplex analysis on a Luminex platform. As shown in FIG. 6A-FIG. 6H, DCP-001 was found to stimulate the secretion of various proinflammatory cytokines (IL-1β, GM-CSF, IFN-γ, IL-2, TNF-α, and IL-6) and chemokines (IL-8 and RANTES) in PBMC. Data represent 12 independent experiments and are expressed as mean±SEM.

1. A pharmaceutical composition comprising an isolated modified cell of leukemic origin comprising a downregulated CD47 pathway, and a pharmaceutically acceptable excipient.

2. The pharmaceutical composition of claim 1, wherein the downregulated CD47 pathway is a result of the depletion and/or inhibition of a member of the CD47 pathway, optionally wherein:
the member of the CD47 pathway is CD47;
the downregulated CD47 pathway is the result of the depletion and/or inhibition of CD47 and/or a member of the CD47 pathway; or
the downregulated CD47 pathway is mediated by an agent that depletes and/or inhibits CD47 and/or a member of the CD47 pathway.

3-5. (canceled)

6. The pharmaceutical composition of claim 2, wherein the agent that depletes CD47 and/or a member of the CD47 pathway is selected from the group consisting of an antibody, a small molecule, a small RNA, or an engineered nuclease system, optionally wherein:
the antibody is an anti-CD47 antibody;
the small RNA is a small interfering RNA (siRNA) or a microRNA (miRNA); and/or
the engineered nuclease system is selected from the group consisting of a meganuclease system, a zinc finger nuclease (ZFN) system, a transcription activator-like effector nuclease (TALEN) system, and a CRISPR system, optionally wherein:
the engineered nuclease system mediates an insertion and/or deletion in a CD47 gene locus and/or the gene locus of a member of the CD47 pathway of the modified cell;
the engineered nuclease system mediates an insertion and/or deletion in a CD47 gene locus of the modified cell; and/or
the engineered nuclease system is a CRISPR system.

7-12. (canceled)

13. A pharmaceutical composition comprising:
a modified cell of leukemic origin comprising an insertion and/or deletion in a CD47 gene locus, wherein the insertion and/or deletion in the CD47 gene locus results in downregulated expression of CD47;
a modified cell of leukemic origin and an anti-CD47 antibody; or
a modified cell of leukemic origin comprising a downregulated CD47 pathway and an anti-CD47 antibody.

14. The pharmaceutical composition of claim 13, wherein the insertion and/or deletion in a CD47 gene locus is mediated by the repair of a double strand break in the CD47 gene locus, optionally wherein:
the insertion and/or deletion in the CD47 gene locus is mediated by an engineered nuclease system,
the engineered nuclease system is selected from the group consisting of a meganuclease system, a zinc finger nuclease (ZFN) system, a Transcription activator-like effector nuclease (TALEN) system, and a CRISPR system,
the engineered nuclease system is a CRISPR system; and/or
the repair is via non-homologous end joining (NHEJ) and homology directed repair (HDR).

15-18. (canceled)

19. The pharmaceutical composition of claim 13, further comprising:
an agent that depletes and/or inhibits CD47 and/or a member of the CD47 pathway, optionally wherein the agent that depletes and/or inhibits CD47 and/or a member of the CD47 pathway is an anti-CD47 antibody; and/or
a pharmaceutically acceptable excipient, optionally wherein the pharmaceutical composition comprises a cryopreservation agent.

20-24. (canceled)

25. The pharmaceutical composition of claim 1, wherein the modified cell comprises at least one tumor associated antigen or a nucleic acid encoding at least one tumor associated antigen, wherein the tumor associated antigen is selected from the group consisting of WT-1, MUC-1, RHAMM, PRAME, p53, and Survivin, optionally wherein the modified cell:
comprises WT-1, MUC-1, PRAME, and Survivin,
comprises an exogenous antigen, wherein the exogenous antigen is a tumor-associated antigen;
comprises a dendritic cell phenotype;
comprises a mature dendritic cell phenotype;
comprises a genetic aberration between chromosome 11p15.5 to 11p12, wherein the genetic aberration encompasses about 16 Mb of genomic regions;
is CD34-positive, CD1a-positive, and CD83-positive;
expresses a cell surface marker selected from the group consisting of DC-SIGN, Langerin, CD80, CD86, CD70, CD40, and any combination thereof;
is CD34-positive, CD la-positive, CD83-positive, CD80-positive, CD86-positive, and CD40-positive;
is CD14-negative;
is derived from the DCOne cell line;
is non-proliferating; or
has been irradiated.

26-39. (canceled)

40. A method of producing a modified cell of leukemic origin comprising a downregulated CD47 pathway, comprising:
incubating a precursor cell under conditions that allow for the differentiation of the precursor cell into an immature cell; and
incubating the immature cell in the presence of an agent that depletes and/or inhibits CD47 and/or a member of the CD47 pathway, and under conditions that allows for the maturation of the immature cell, thereby producing the modified cell comprising a downregulated CD47 pathway.

41. The method of claim 40, wherein the agent that depletes and/or inhibits CD47 and/or a member of the CD47 pathway is selected from the group consisting of an antibody, a small molecule, a small RNA, or an engineered nuclease system, optionally wherein:
the antibody is an anti-CD47 antibody;
the small RNA is a small interfering RNA (siRNA) or a microRNA (miRNA); or the engineered nuclease system mediates an insertion and/or deletion in a CD47 gene locus and/or the gene locus of a member of the CD47 pathway of the modified cell, optionally wherein the engineered nuclease system:
mediates an insertion and/or deletion in a CD47 gene locus of the modified cell;
is selected from the group consisting of a meganuclease system, a zinc finger nuclease (ZFN) system, a transcription activator-like effector nuclease (TALEN) system, and a CRISPR system; and/or
is a CRISPR system.

42-47. (canceled)

48. A pharmaceutical composition comprising the modified cell produced by the method of claim 40, optionally further comprising a pharmaceutiaccly accptable excipient and/or a cryopreservation agent.

49. (canceled)

50. (canceled)

51. A method of enhancing an immune response in a subject in need thereof or treating or preventing cancer in a subject in need thereof, comprising administering to the subject an effective amount of the composition of claim 1.

52. (canceled)

53. A method of enhancing an immune response in a subject in need thereof, comprising:
administering to the subject a first composition comprising a modified cell of leukemic origin comprising a downregulated CD47 pathway; or
administering to the subject a first composition comprising a modified cell of leukemic origin, and an agent that depletes and/or inhibits CD47 and/or a member of the CD47 pathway.

54. The method of claim 53, wherein the first composition further comprises an agent that depletes and/or inhibits CD47 and/or a member of the CD47 pathway, optionally wherein:
the method further comprises administering to the subject an effective amount of a second composition comprising an agent that depletes and/or inhibits CD47;
the first composition and the second composition are administered simultaneously;
the first composition is administered before the second composition; or
the first composition is administered after the second composition.

55-59. (canceled)

60. The method of claim 53, wherein the modified cell comprises at least one tumor associated antigen or a nucleic acid encoding the tumor associated antigen, wherein the tumor associated antigen is associated with the tumor in the subject, optionally wherein the modified cell comprises at least one tumor associated antigen or a nucleic acid encoding the tumor associated antigen, wherein the tumor associated antigen is not associated with the tumor in the subject.

61. (canceled)

62. The method of claim 54, wherein:
the first composition and/or the second composition is administered via a route selected from the group consisting of intramuscular, subcutaneous, intravenous, intraarterial, intraperitoneal, intrasternal, intradermal, transcutaneous, transdermal, delivery to the interstitial space of a tissue, and delivery to a non-tumor tissue;
the first composition and/or the second composition is administered intravenously;
the first composition and/or the second composition is administered intradermally, the first composition and/or the second composition is administered intramuscularly; or
the first composition and/or the second composition is administered intratumorally.

63. (canceled)

64. The method of claim 62, wherein:
the first composition and/or the second composition is prepared for intravenous administration;
the first composition and/or the second composition comprises a diluent or solvent acceptable for intravenous administration;
the first composition and/or the second composition is prepared for intradermal administration, optionally wherein the first composition and/or the second composition comprises a diluent or solvent acceptable for intradermal administration;
the first composition and/or the second composition is prepared for intramuscular administration, optionally wherein the first composition and/or the second composition comprises a diluent or solvent acceptable for intramuscular administration; or
d) the first composition and/or the second composition is prepared for intratumoral administration, optionally wherein the first composition and/or the second composition comprises a diluent or solvent acceptable for intratumoral administration.

65-74. (canceled)

75. The method of claim 54, wherein:
the agent that depletes and/or inhibits CD47 is selected from the group consisting of an antibody, a small molecule, a small RNA, or an engineered nuclease system,
the antibody is an anti-CD47 antibody,
the small RNA is a small interfering RNA (siRNA) or a microRNA (miRNA),
the engineered nuclease system mediates an insertion and/or deletion in a CD47 gene locus and/or the gene locus of a member of the CD47 pathway of the modified cell, optionally wherein the engineered nuclease system:
mediates an insertion and/or deletion in a CD47 gene locus of the modified cell,
is selected from the group consisting of a meganuclease system, a zinc finger nuclease (ZFN) system, a transcription activator-like effector nuclease (TALEN) system, and a CRISPR system; or
is a CRISPR system.

76-81. (canceled)

82. The method of claim 54, wherein the agent that depletes and/or inhibits CD47 comprises a viral vector comprising a nucleic acid encoding an anti-CD47 antibody, a CD47-targeting small RNA, or a CD47-targeting engineered nuclease system, optionally wherein:
the viral vector is derived from a virus selected from the group consisting of a retrovirus, an adenovirus, an adeno-associated virus, and a herpes simplex virus;
the CD47-targeting small RNA is a small interfering RNA (siRNA) or a microRNA (miRNA); and/or
wherein the CD47-targeting engineered nuclease system mediates an insertion and/or deletion in a CD47 gene locus of the modified cell, optionally wherein the CD47-targeting engineered nuclease system is selected from the group consisting of a meganuclease system, a ZFN system, a TALEN system, and a CRISPR system, or is a CRISPR system.

83-87. (canceled)

88. The method of claim 53, wherein the modified cell comprises at least one tumor associated antigen or a nucleic acid encoding at least one tumor associated antigen, wherein the tumor associated antigen is selected from the group consisting of WT-1, MUC-1, RHAMM, PRAME, p53, and Survivin, optionally wherein the modified cell:
comprises WT-1, MUC-1, PRAME, and Survivin;
comprises an exogenous antigen, wherein the exogenous antigen is a tumor-associated antigen;
comprises a dendritic cell phenotype;
comprises a mature dendritic cell phenotype;
comprises a genetic aberration between chromosome 11p15.5 to 11p12, wherein the genetic aberration encompasses about 16 Mb of genomic regions;
is CD34-positive, CD1a-positive, and CD83-positive;
expresses a cell surface marker selected from the group consisting of DC-SIGN, Langerin, CD80, CD86, CD70, CD40, and any combination thereof;
is CD34-positive, CD1a-positive, CD83-positive, CD80-positive, CD86-positive, and CD40-positive;
is CD14-negative;
is derived from the DCOne cell line;
is non-proliferating; or
has been irradiated.

89102. (canceled)

103. The method of claim 53, wherein the subject has previously suffered from the cancer, the subject has previously received treatment for the cancer, or the subject is suffering from relapse of the cancer, optionaly wherein:
the cancer is a tumor or a solid tumor, optionally wherein the solid tumor is selected from the group consisting of a sarcoma, a carcinoma, and a lymphoma; and/or
the subject is a human, a domesticated animal, or an animal suitable for veterinary healthcare.

104-110. (canceled)
